(19) United States
(12) Patent Application Publication
(10) Pub. No.: US 2013/0158348 A1
(43) Pub. Date: Jun. 20, 2013

(54) INTRODUCER FOR AN INTERNAL MAGNETIC CAMERA

(71) Applicants: Rudolph H. Nobis, Mason, OH (US); James T. Spivey, Cincinnati, OH (US); Alexander P. Kondor, Cincinnati, OH (US); Kempton K. Carroll, II, Cincinnati, OH (US); Christopher J. Hess, Cincinnati, OH (US); Sean P. Conlon, Loveland, OH (US); David B. Griffith, Cincinnati, OH (US)

(72) Inventors: Rudolph H. Nobis, Mason, OH (US); James T. Spivey, Cincinnati, OH (US); Alexander P. Kondor, Cincinnati, OH (US); Kempton K. Carroll, II, Cincinnati, OH (US); Christopher J. Hess, Cincinnati, OH (US); Sean P. Conlon, Loveland, OH (US); David B. Griffith, Cincinnati, OH (US)

(73) Assignee: Ethicon Endo-Surgery, Inc., Cincinnati, OH (US)

(21) Appl. No.: 13/325,791

(22) Filed: Dec. 14, 2011

(51) Int. Cl. A61B 1/04

(52) U.S. Cl. 600/109

(57) ABSTRACT

An introducer for an internal magnetic camera is provided. The introducer may be arranged to enable the camera to be placed inside of a body cavity through an otomy or incision and then oriented next to the tissue of the body cavity opposite an External Control Unit (“ECU”) outside of the body cavity. The camera may be retained in the introducer by a magnet or a magnetic material, by a selectively engageable retainer, or by covering the camera within a cavity. The camera may be retained in the introducer by a latch. The camera may include a keyed surface that only can align with a matching keyed surface on the retainer in a single orientation. The introducer may include a spring to eject the camera from the introducer when the retainer is disengaged.

### BACKGROUND

Magnetic anchoring and guidance systems (MAGS) have been developed for use in minimally invasive procedures. MAGS include an internal device attached in some manner to a surgical instrument, laparoscope or other camera or viewing device, and an external hand held device or external control unit (“ECU”) for controlling the movement of the internal device. Each of the external and internal devices has magnets which are magnetically coupled to each other across, for example, a patient's abdominal wall. In the current systems, the external magnet may be adjusted by varying the height of the external magnet.

The foregoing discussion is intended only to illustrate various aspects of the related art in the field of the invention at the time, and should not be taken as a disavowal of claim scope.

### SUMMARY

In various embodiments, an introducer for an internal magnetic camera is provided. The introducer enables insertion of the camera into a body cavity through an incision or otomy and positions the camera adjacent the tissue of the body cavity so that it may be magnetically coupled across tissue to an External Control Unit.

In at least one embodiment, the introducer may include a shaft with a hollow portion for receiving the camera therein. The hollow portion may include magnets or a magnetic material that attracts magnets in the camera. The hollow portion also may include a longitudinal slot for receiving a tether of the camera. The shaft may be coupled to a curved handle.

In at least one embodiment, the introducer may be incorporated into a trocar. The camera may be placed within a trocar cannula tube after a trocar obturator has been removed. The camera may be placed within an obturator of the trocar. The trocar may have a sharp tip for cutting an incision or otomy in the tissue of the body cavity. The trocar may have a blunt tip that guides the obturator between tissues. The tip may be transparent to provide an unimpeded view for the lens of the camera through the tip. The tip may be opaque and include a window port through which the camera lens may view the tissue.

In at least one embodiment, the introducer may include a movable latch or retainer for selectively engaging the camera to hold the camera within a port or cavity of the introducer when in use. The latch or retainer may be moved between engaged and disengaged positions by a plunger or a trigger. The latch or retainer may be biased in the engaged or the disengaged position by a spring. The camera may be biased out of the port or cavity by a spring.

In at least one embodiment, the introducer may include a jointed handle that includes at least one elbow defining an angle between a distal end of the introducer that carries the camera and portion of the introducer more proximate to a proximal end of the introducer. The elbow may enable the distal end of the introducer to place the camera closer to the tissue of the body cavity without twisting or contorting the tissue.

In at least one embodiment, the introducer may include an open region at the distal end of a hollow shaft that is sized to conform to an outer surface of a trocar such that the distal end of the introducer can slide next to a trocar through an otomy without overstretching the otomy. The introducer may include a ram rod for pushing the camera out of the hollow shaft after the introducer is inserted into the otomy.

In at least one embodiment, the introducer may include two concentric shafts. The inner shaft includes a port or cavity for carrying a camera. The outer shaft slides longitudinally relative to the inner shaft and may selectively cover and uncover the port or cavity. The inner shaft also may include a tip for creating an otomy or for guiding the shafts through an existing otomy and into a body cavity. Once inside the body cavity, the outer shaft may be slid to expose the cavity and the camera, enabling the camera to be extracted.

In at least one embodiment, the introducer may include a channel that comprises an arcuate distal end for holding a camera. The introducer may include a plunger that slides relative to the channel and, in use, may push the camera out of the introducer. The plunger may include a slot that may carry tethers for the camera. The plunger may include a seal over the slot to prevent insufflation gases from escaping from the body cavity when in use. The seal may include apertures sized to enable the tethers to escape from the plunger. The seal also may include a slit for enabling the tethers to be removed from the plunger when the camera is removed from the introducer.

### BRIEF DESCRIPTION OF THE DRAWINGS

Various features of the embodiments described herein are set forth with particularity in the appended claims. The various embodiments, however, both as to organization and methods of operation, together with advantages thereof, may be understood in accordance with the following description taken in conjunction with the accompanying drawings as follows.

FIG. 1 is a side view of an embodiment of an introducer;

FIG. 2 is a perspective view of one side of the end of the introducer depicted in FIG. 1;

FIG. 3 is a perspective view of the opposite side of the end of the introducer depicted in FIG. 1;

FIG. 4 is a side view of another embodiment of an introducer with an obturator;

FIG. 5 is a perspective view of the introducer depicted in FIG. 4;

FIG. 6 is a perspective view of another embodiment of an introducer having a sharp distal tip and showing the camera removed;

FIG. 7 is a perspective view of the introducer depicted in FIG. 6 showing the camera positioned in the introducer;

FIG. 8 is a perspective detail view of the introducer depicted in FIG. 7;

FIG. 9 is a perspective view of an embodiment of an introducer having a blunt distal end;

FIG. 10 is a perspective view of the embodiment depicted in FIG. 9 showing the camera positioned in the introducer;

FIG. 11 is a perspective detail view of the introducer depicted in FIG. 9;

FIG. 12 is a perspective detail view of the distal tip of the introducer depicted in FIG. 9;

FIG. 13 is a perspective view of another embodiment of an introducer;

FIG. 14 is a side cross-sectional view of the introducer of FIG. 13 releasing the camera from the introducer;

FIG. 15 is a perspective view of the introducer of FIG. 13;

FIG. 16 is a perspective view of the introducer of FIG. 13 showing the camera removed from the introducer;

FIG. 17 is a perspective view of the lower side of the introducer of FIG. 13 showing the camera positioned in the introducer;

FIG. 18 is a perspective view of the introducer of FIG. 13 showing the camera removed from the introducer;

FIG. 19A is a perspective view of another embodiment of an introducer;

FIG. 19B is a perspective view of the introducer of FIG. 19A with an engagement member positioned in the introducer;

FIG. 19C is a top view of the introducer of FIG. 19A with the engagement member in a first position;

FIG. 19D is a top view of the introducer of FIG. 19A with the engagement member in a second position;

FIG. 20 is a perspective detail view of the distal end of the introducer of FIG. 19;

FIG. 21 is a top view of the distal end of the introducer of FIG. 19;

FIG. 22 is a perspective detail view of the introducer of FIG. 19 inserted through a wall of tissue showing the camera removed from the introducer;

FIG. 23 is a perspective detail view of the introducer of FIG. 19;

FIG. 24 is a perspective view of another embodiment of an introducer with the ram rod removed;

FIGS. 25A-25I illustrate method steps that may be performed with the introducer of FIG. 24;

FIG. 26 is a perspective view of another embodiment of an introducer with the inner second shaft removed;

FIGS. 27A-27D illustrate method steps that may be performed with the introducer of FIG. 26;

FIG. 28 is a perspective view of another embodiment of an introducer;

FIG. 29 is a perspective view of a shell portion of the introducer depicted in FIG. 28;

FIG. 30 is a perspective view of a shaft and seal portion of the introducer depicted in FIG. 28;

FIG. 31 is a perspective view of a seal portion of the introducer depicted in FIG. 28;

FIG. 32 is a perspective view of combined shaft and seal portions of the introducer depicted in FIG. 28 with a camera and tethers in place;

FIG. 33 is a perspective view of an embodiment of a heating element that may be used in conjunction with an introducer; and

FIG. 34 is a perspective view of the heating element depicted in FIG. 33 attached to the distal tip of an introducer.

Corresponding reference characters indicate corresponding parts throughout the several views. The exemplifications set out herein illustrate various embodiments of the invention, in one form, and such exemplifications are not to be construed as limiting the scope of the invention in any manner.

### DETAILED DESCRIPTION

Numerous specific details are set forth to provide a thorough understanding of the overall structure, function, manufacture, and use of the embodiments as described in the specification and illustrated in the accompanying drawings. It will be understood by those skilled in the art, however, that the embodiments may be practiced without such specific details. In other instances, well-known operations, components, and elements have not been described in detail so as not to obscure the embodiments described in the specification. Those of ordinary skill in the art will understand that the embodiments described and illustrated herein are non-limiting examples, and thus it can be appreciated that the specific structural and functional details disclosed herein may be representative and do not necessarily limit the scope of the embodiments, the scope of which is defined solely by the appended claims.

In describing and claiming the present invention, the following terminology will be used in accordance with the definitions set out below.

Reference throughout the specification to “various embodiments,” “some embodiments,” “one embodiment,” or “an embodiment”, or the like, means that a particular feature, structure, or characteristic described in connection with the embodiment is included in at least one embodiment. Thus, appearances of the phrases “in various embodiments,” “in some embodiments,” “in one embodiment,” or “in an embodiment”, or the like, in places throughout the specification are not necessarily all referring to the same embodiment. Furthermore, the particular features, structures, or characteristics may be combined in any suitable manner in one or more embodiments. Thus, the particular features, structures, or characteristics illustrated or described in connection with one embodiment may be combined, in whole or in part, with the features structures, or characteristics of one or more other embodiments without limitation.

It will be appreciated that the terms “proximal” and “distal” may be used throughout the specification with reference to a clinician manipulating one end of an instrument used to treat a patient. The term “proximal” refers to the portion of the instrument closest to the clinician and the term “distal” refers to the portion located farthest from the clinician. It will be further appreciated that for conciseness and clarity, spatial terms such as “vertical,” “horizontal,” “up,” and “down” may be used herein with respect to the illustrated embodiments. However, surgical instruments may be used in many orientations and positions, and these terms are not intended to be limiting and absolute.

As used herein, the term “biocompatible” includes any material that is compatible with the living tissues and system(s) of a patient by not being substantially toxic or injurious and not known to cause immunological rejection. “Biocompatibility” includes the tendency of a material to be biocompatible.

As used herein, the term “patient” refers to any human or animal on which a suturing procedure may be performed. As used herein, the term “internal site” of a patient means a lumen, body cavity or other location in a patient's body including, without limitation, sites accessible through natural orifices or through incisions.

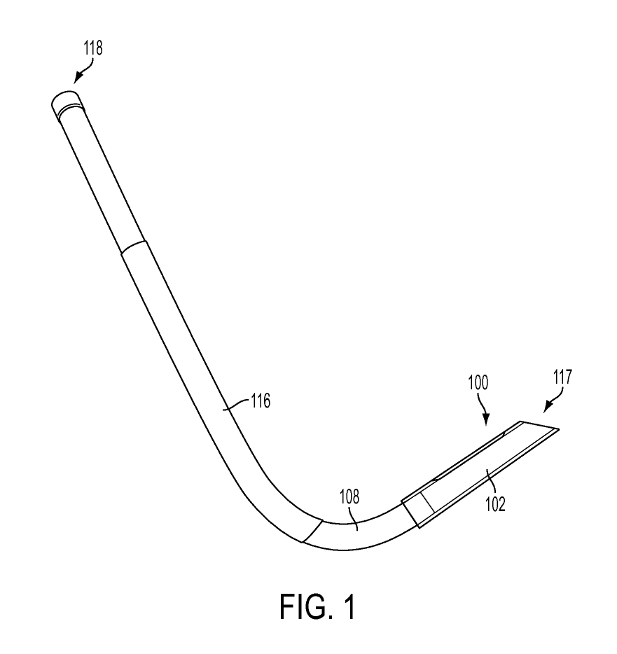

FIG. 1 shows an embodiment of an introducer 100 for a steerable internal magnetic camera (referred to herein as a “camera”). Examples of such cameras are described in NEED APP. SER. NO., FILING DATE, & TITLE, the relevant portions of which are incorporated herein by reference. The introducer 100 includes a hollow tube 102 at a distal end 117. The hollow tube 102 is attached to a handle that includes a first proximal portion 116 and a second portion 108 distal to the first portion. The transition between the first and second portions 116 and 118 forms a curved portion, which allows the distal end 117 of the introducer 100, specifically the hollow tube 102 carrying a camera (not shown), to be inserted through an otomy or incision, for example, in the abdominal wall of a patient. After the hollow tube 102 is inserted into a body cavity through the otomy, the handle is rotated so that the hollow tube 102 is approximately parallel to the abdominal wall.

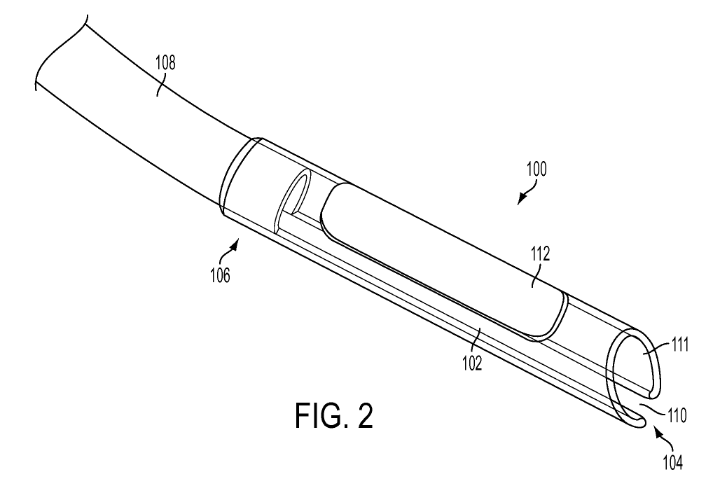

FIG. 2 shows the hollow tube 102 of the introducer 100. The hollow tube 102 includes a distal end 104 and a proximal end 106. The proximal end 106 of the hollow tube 102 is attached to the second portion 108. The distal end 104 includes an opening 111 and a slot 110. The hollow tube 102 may include a magnet 112 on a surface of the hollow tube 102. The magnet 112 may be on an interior surface or an exterior surface of the hollow tube 102, or the magnet 112 may be within layers of the material forming the hollow tube 102. The magnet 112 may be replaced by a material that attracts a magnet, such as, for example, an iron plate. When a camera (not shown) is inserted through the opening 111 at the distal end 104 of the hollow tube 102, magnets in the camera are attracted to the magnet 112 (or the magnetically attractive material), and the camera thereby is held in place in the hollow tube 102. Tethers (not shown) attached to the camera pass through the slot 110 when the camera is positioned in the hollow tube 102.

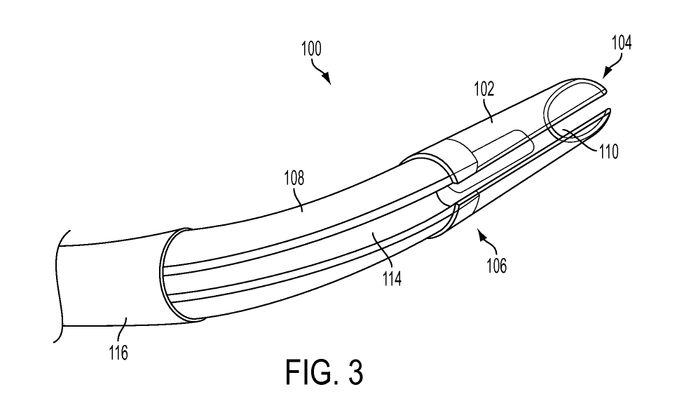

FIG. 3 shows the second portion 108 of the handle in more detail. In certain embodiments, the slot 110 in the hollow tube 102 may span the length of the hollow tube 102, and the second portion 108 of the handle may include a slot 114 that continues the slot 110 in the hollow tube 102. The tether (not shown) of an internal magnetic camera may be fed through the slots 110 and 114. When the introducer is inserted into a body cavity through an otomy, at least a portion of the slot 114 in the second portion 108 of the handle remains outside of the otomy, enabling the tether to exit the body cavity.

After the introducer 100 carrying a camera (not shown) is placed inside the abdominal cavity and has been rotated to bring the hollow tube 102 parallel with the abdominal wall, an Electronic Control Unit (“ECU”) (not shown), may be placed on the exterior surface of the abdomen adjacent to the hollow tube 102. Examples of ECUs are described in NEED APP. SER. NO., FILING DATE, & TITLE, the relevant portions of which are incorporated herein by reference. When the ECU is energized, a magnetic field created by the ECU creates magnetic attraction between the camera and the ECU that overpowers the magnetic attraction between the magnet 112 and the camera. The ECU then is capable of removing the camera from the hollow tube 102. The tether (not shown) follows the camera out of the hollow tube 102 via slots 114 and 110. After the camera and the tether have been removed from the hollow tube 102, the introducer 100 may be removed from the abdominal cavity. When the surgeon is ready to remove the camera from the body cavity, the introducer 100 can be reinserted through the otomy and the camera can be maneuvered close to the distal end 104 of the hollow tube 102 by the ECU. When the ECU is de-energized or turned off, the magnetic attraction between the magnet 112 in the hollow tube 102 and the camera may bring the two together such that the camera is drawn back into the hollow tube 112. The surgeon then can remove the introducer 100 and the camera.

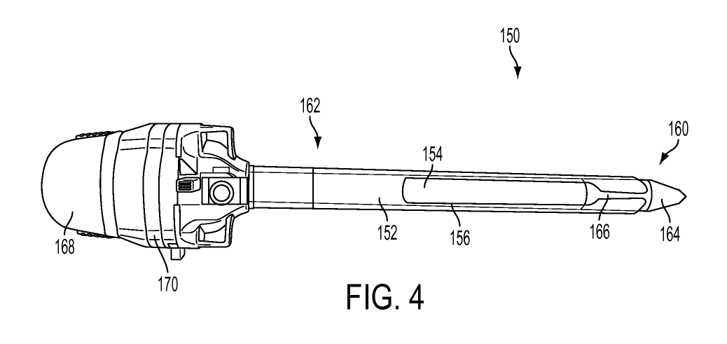

In various embodiments, an introducer may be incorporated into a trocar. The introducer 150 may be incorporated into a trocar cannula tube 152, which may be attached to a cannula handle 170 at a proximal end 162. FIG. 4 shows an obturator shaft 154 passing through the cannula tube 152, which may include an obturator grip 168 adjacent to the cannula handle 170 and an obturator tip 164 protruding from a distal end 160 of the cannula tube 152. The obturator tip 164 may include a sharp tip and one or more cutting edges for piercing tissue such as, for example, an abdominal wall, to form an otomy or incision. After the trocar has formed the otomy, it may be removed from the body cavity. After being removed, the obturator shaft 154 and obturator tip 164 may be removed from the cannula tube 152.

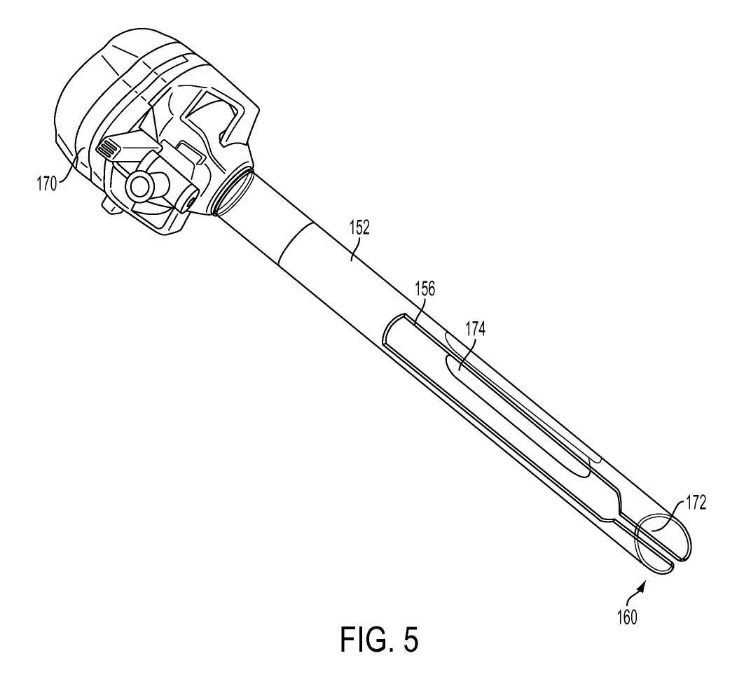

When the obturator shaft 154 and obturator tip 164 are removed from the cannula tube 152 (as shown in FIG. 5), a camera (not shown) may be inserted into the cannula tube 152. The camera may be inserted into a port 156 on a side of the cannula tube 152. The camera also may be inserted into the cannula tube 152 via an opening 172 at the distal end 160 of the cannula tube 152. A magnet or metal plate 174 is located on the cannula tube 152 wall at a location generally opposite to the port 156. As the camera is inserted into the port 156, magnets in the camera are attracted to the metal plate 174, and magnetic attraction between the magnets in the camera and the metal plate 174 hold the camera in place until a surgeon is ready to extract the camera with an ECU. The cannula tube 152 may include a slot 166 at the distal end 160 that accommodates a tether (not shown) of the camera.

After the camera (not shown) is loaded into the cannula tube 152, the surgeon reinserts the cannula tube 152 through the otomy in the patient. The surgeon then torques the cannula tube 152 about the point where it enters the otomy such that the cannula handle 170 moves towards the exterior surface of the tissue (not shown) and the distal end 160 of the cannula tube 152, which is inside of the body cavity, moves towards the interior surface of the tissue. In the rotated configuration, the camera is proximate and parallel to the interior surface of the tissue. A surgeon then can place an ECU (not shown) on the exterior surface of the tissue near the location of the camera and, as described above, use the ECU to extract the camera from the cannula tube 152 via an opening 172 at the distal end 160. The camera's tether (not shown) may exit the cannula tube 152 via the slot 166. After the camera is extracted from the cannula tube 152, the surgeon may reorient the cannula tube 152 to its non-rotated configuration and remove the cannula tube 152 from the otomy. The surgeon also may leave the cannula tube 152 in place in the otomy and introduce additional endoscopic instruments through the cannula tube 152.

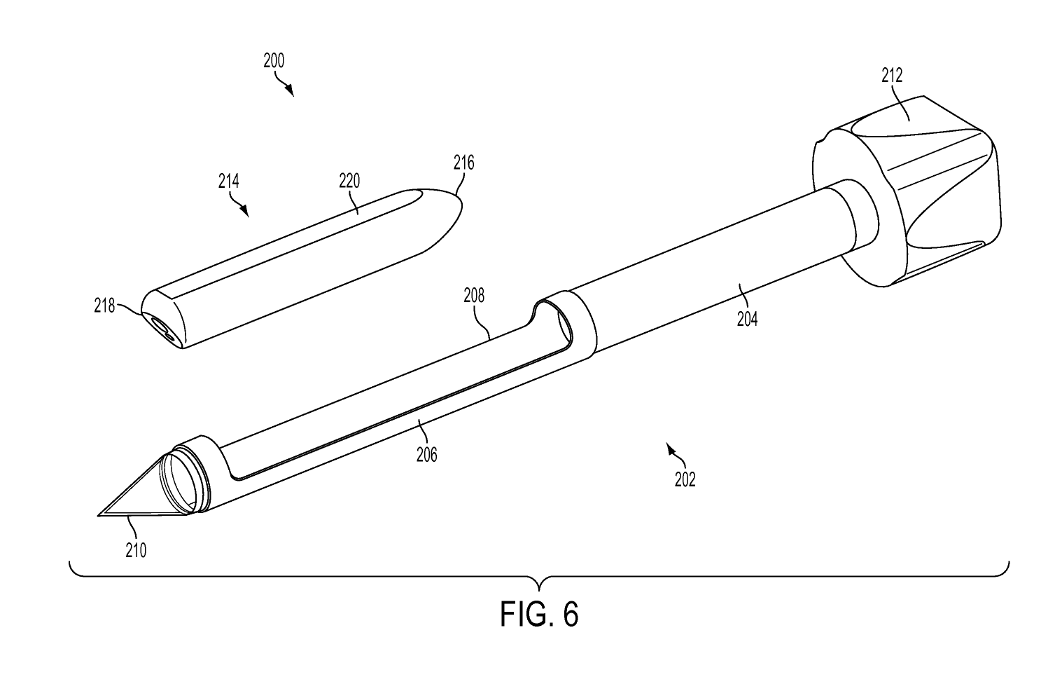

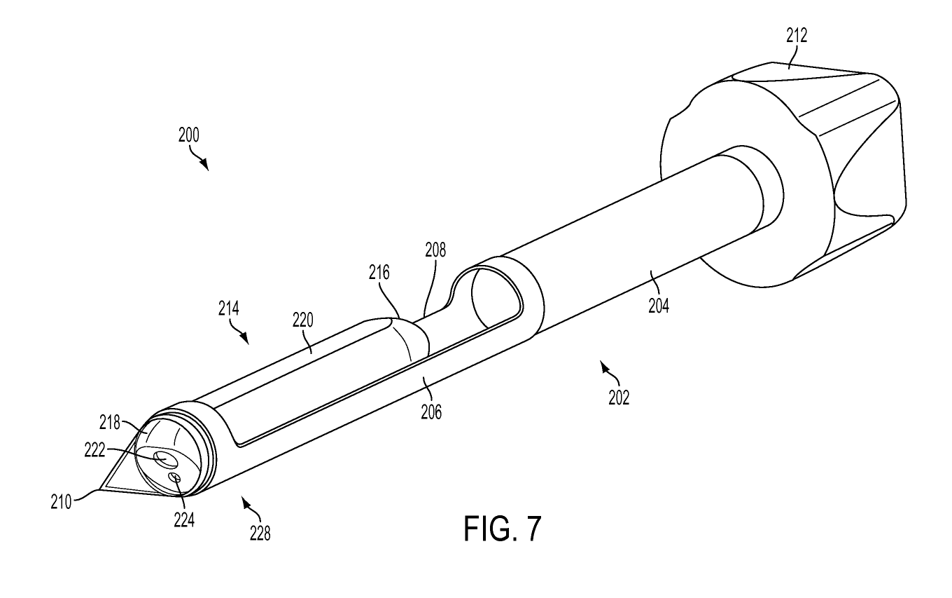

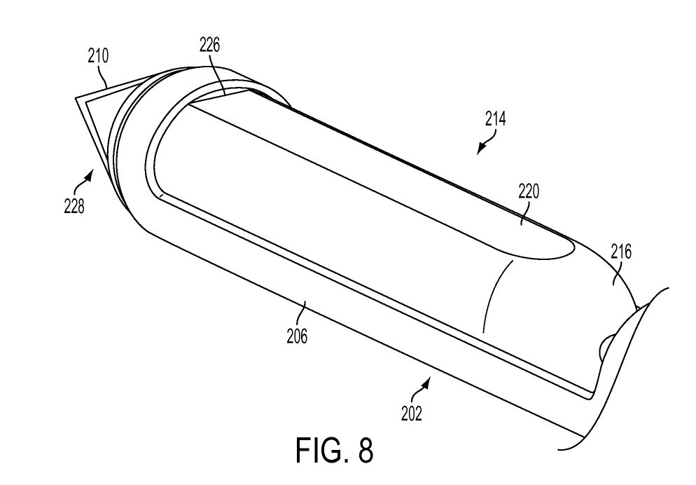

FIGS. 6-8 show another embodiment of an introducer 200 for a camera 214. The introducer 200 may include a trocar shaft 202 having a proximal portion 204 and a distal portion 206. The distal portion 206 is generally hollow and includes a port 208 configured to receive the camera 214. The distal portion 206 may comprise a metal plate or magnet (not shown) in the port that magnetically attracts magnets (not shown) in the camera 214. The trocar shaft 202 may include a handle 212 at its proximal end and a tip 210 at its distal end. The tip 210 may be sharp to enable the tip 210 to pierce through tissue, for example, to create an otomy in an abdominal wall (not shown). For example, the tip 210 may comprise a sharp point and one or more sharp cutting edges that pierce and incise tissue, respectively. The tip 210 also may comprise a transparent plastic that enables the camera 214 to see through it. FIG. 7 shows the camera 214 in the introducer 200 with a distal end 218 of the camera inserted in a distal cavity 228 of the distal portion 206 of the trocar shaft 202. The distal cavity 228 comprises a hollow region that may be open to (or in fluid communication with) the tip 210. The camera port 224 located on the distal end 218 of the camera 214 can see through the open end of the distal cavity 228 and through the transparent tip 210. In this configuration, the camera 214 may be used to visualize a body cavity into which the trocar assembly 200 is being inserted. The distal cavity 228 also comprises a mating surface, such as, for example, a flat 226 (shown in FIG. 8), that corresponds to a complimentary surface, such as, for example, a similar flat 220, on the camera 214. To insert the camera 214 into the port 208, the camera 214 must be oriented so that its mating surface, flat 220, is aligned with the complimentary mating surface, flat 226, of the distal cavity 228. Likewise, the flat 226 of the distal cavity 228 mating with the flat 220 on the camera 214 prevents the camera 214 from rotating about its longitudinal axis relative to the trocar shaft 202 when the camera 214 is installed in the trocar shaft 202. Those skilled in the art will appreciate that other complimentary mating surfaces or other alignment or anti-rotation structures may be used. In various embodiments, the distal cavity is configured to receive a distal end of the camera to secure the camera in position in the distal portion and port of the introducer. The port is longer than the length of the camera so that the camera may be moved proximally, out of the distal cavity when it is to be deployed in position within the body cavity.

After the introducer 200 is inserted into the body cavity (not shown), the camera 214 may be removed from the introducer 200 with an ECU, for example, as described above with respect to FIGS. 4 and 5. Likewise, the camera 214 may be recaptured from the body cavity by the introducer 200 at the end of a surgical procedure as described above.

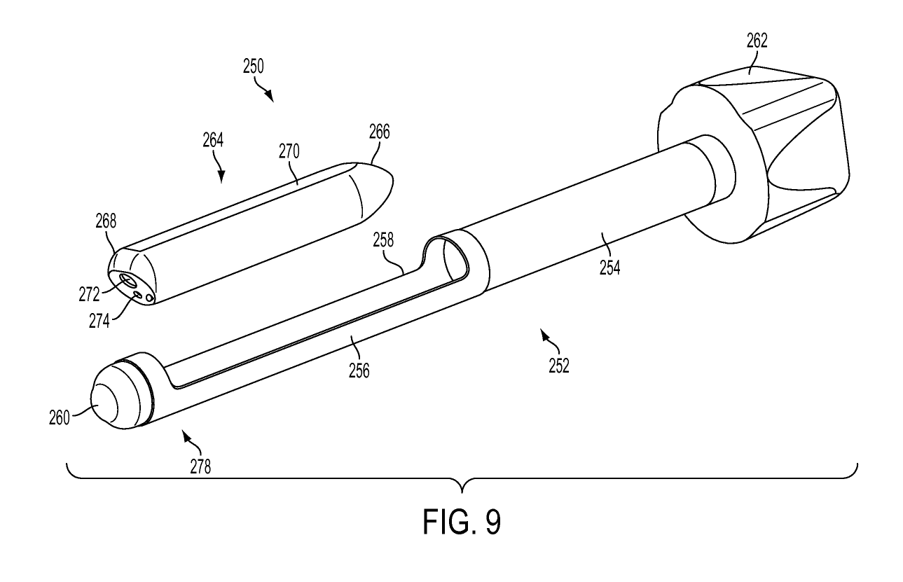

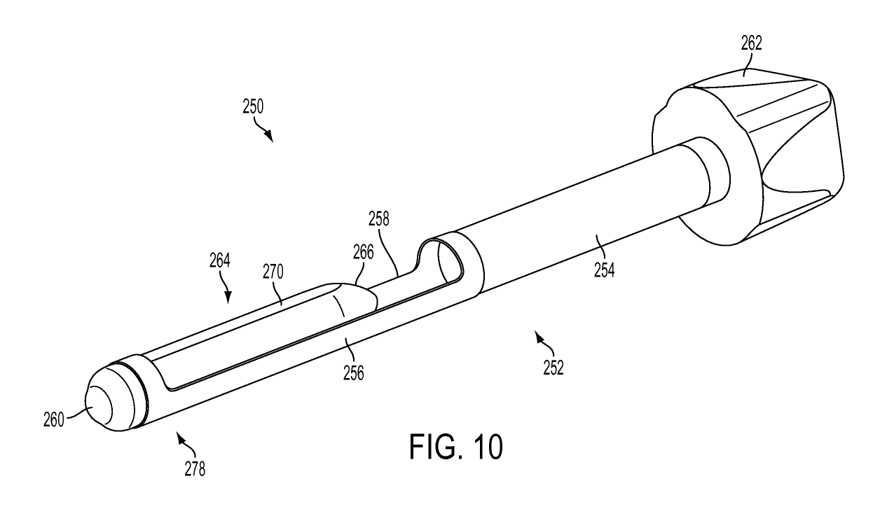

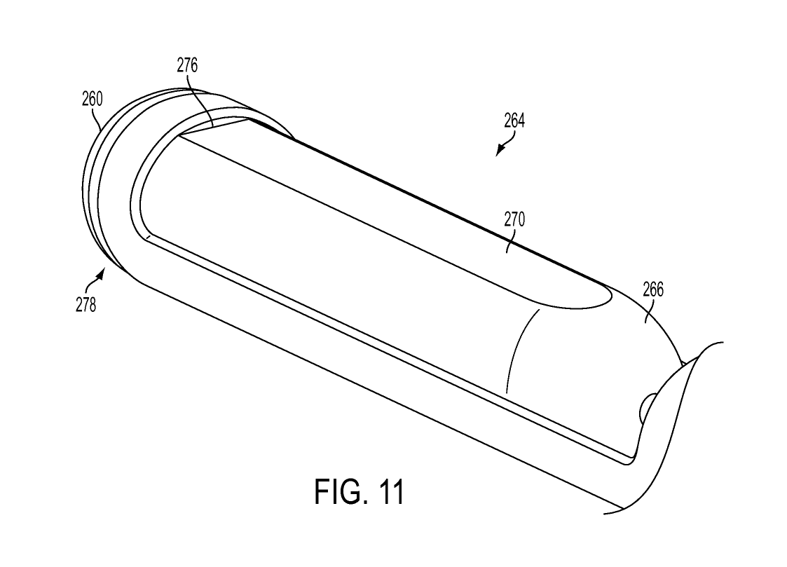

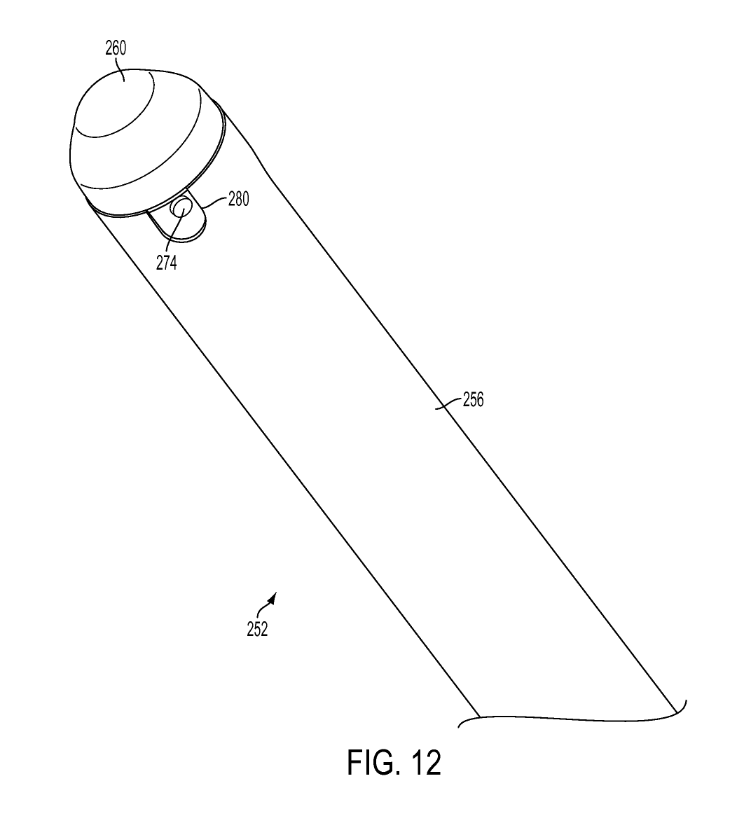

FIGS. 9-12 show another embodiment of an introducer 250 for an internal magnetic camera 264. The introducer 250 is similar to introducer 200 shown in FIGS. 6-8, except that the sharp tip 210 is replaced with a blunt tip 260. The blunt tip 260 may be used to guide the trocar shaft 252 through an already-created otomy (not shown) in a body cavity (not shown). Also, the blunt tip may comprise an opaque material. To enable the camera port 274 of the camera 264 to visualize the body cavity into which the introducer 250 with an opaque tip is being inserted, an aperture 280 (shown in FIG. 12) may be included in the distal portion 256 of the trocar shaft 252. The aperture 280 is oriented relative to the mating surface, for example, flat 276, of the distal cavity 278 such that when the camera 264 is inserted into the introducer 250, and the mating surface of the distal cavity 278 mates with the complimentary surface of the camera 264. The camera port 274 on the camera 264 is aligned with the aperture 274, and an image from the camera 264 can be provided to the surgeon.

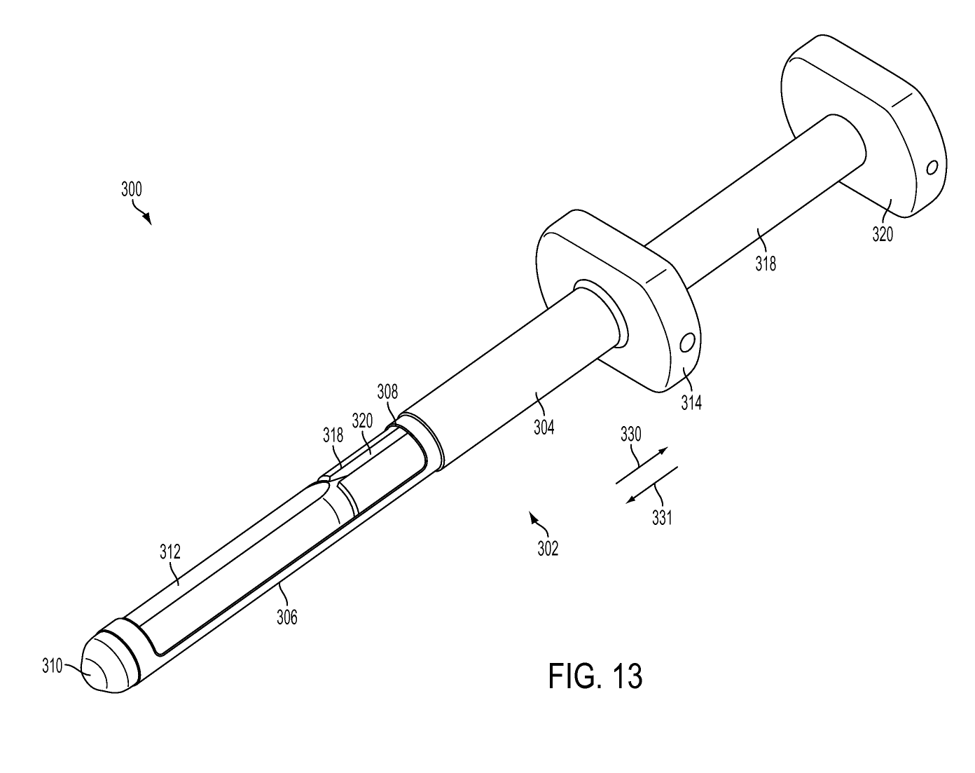

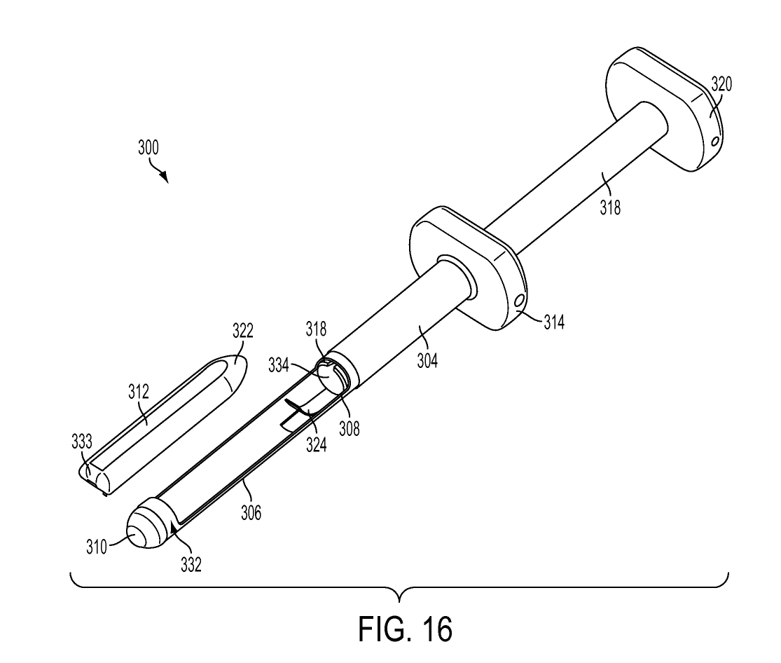

FIGS. 13-18 show another embodiment of an introducer 300 for an internal magnetic camera 312. The introducer 300 includes a shaft 302 that may be generally hollow. The shaft may comprise a single piece or, as shown in FIG. 13, may include multiple sections, such as, for example, a distal section 306 and a proximal section 304. The distal and proximal sections 306 and 304 may be joined by, for example, welding, adhesive, interference fitting, or overmolding. A grip 314 may be attached to the proximal end of the hollow shaft 302. The distal section 306 includes a port 308 configured to receive the camera 312. The distal section 306 may include or have attached to or incorporated therein a metal plate or magnet (not shown) that magnetically attracts magnets (not shown) in the camera 312. The distal section 306 also includes a distal cavity 332 similar to those described above with respect to FIGS. 6-12. FIGS. 13-18 show the introducer 300 having a blunt tip 310. However, the tip 310 may be sharp and transparent, such as, for example, tip 210 shown in FIGS. 6-8.

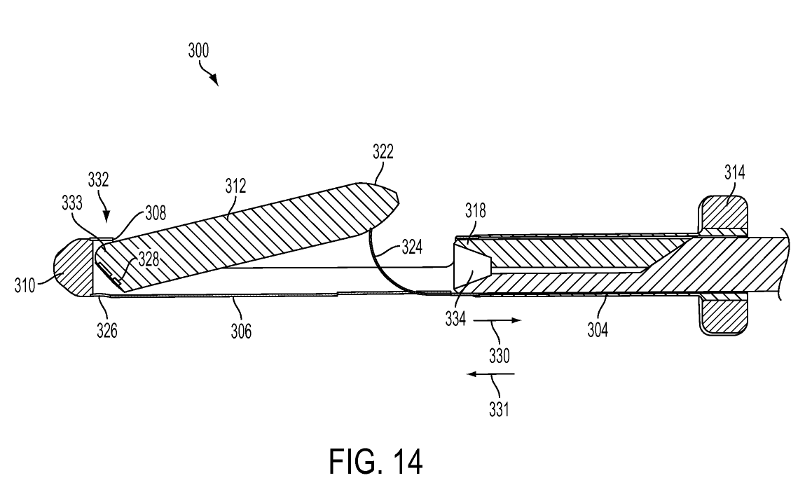

The introducer 300 may include a rod 318 arranged coaxially with shaft 302 and configured to move in a longitudinal direction relative to shaft 302. The rod 318 may be attached to a grip 320 at its proximal end. The rod 318 may be moved relative to shaft 302 by moving grip 320 relative to grip 314. By moving grip 320 towards grip 314, the rod 318 moves in the direction indicated by arrow 331 (shown in FIG. 14) relative to shaft 302. Likewise, moving grip 320 away from grip 314 moves the rod 318 in the direction of arrow 330 (shown in FIG. 14) relative to the shaft 302. A distal end of the rod 318 may be arranged within the hollow shaft 302. The distal end of the rod 318 may include a cavity 334 that mates to a proximal end 322 of the camera 312. The distal end of the rod 318 also includes a slot 320 that can accommodate a tether (not shown) of the camera 312. After the camera 312 is inserted into the port 308, the rod 318 may be moved relative to the shaft 302 in the direction indicated by arrow 331 in FIG. 14. When the rod 318 is moved in the direction of arrow 331, the proximal end 322 of the camera 312 may be captured by the cavity 334 in the distal end of the rod 318, and the distal end 333 of the camera 312 may be captured by the distal cavity 332 of shaft 302, and the camera 312 is thereby secured in the introducer 300.

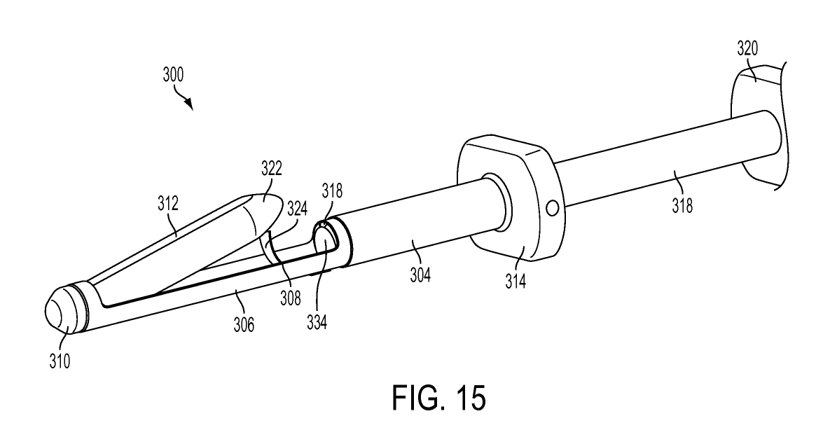

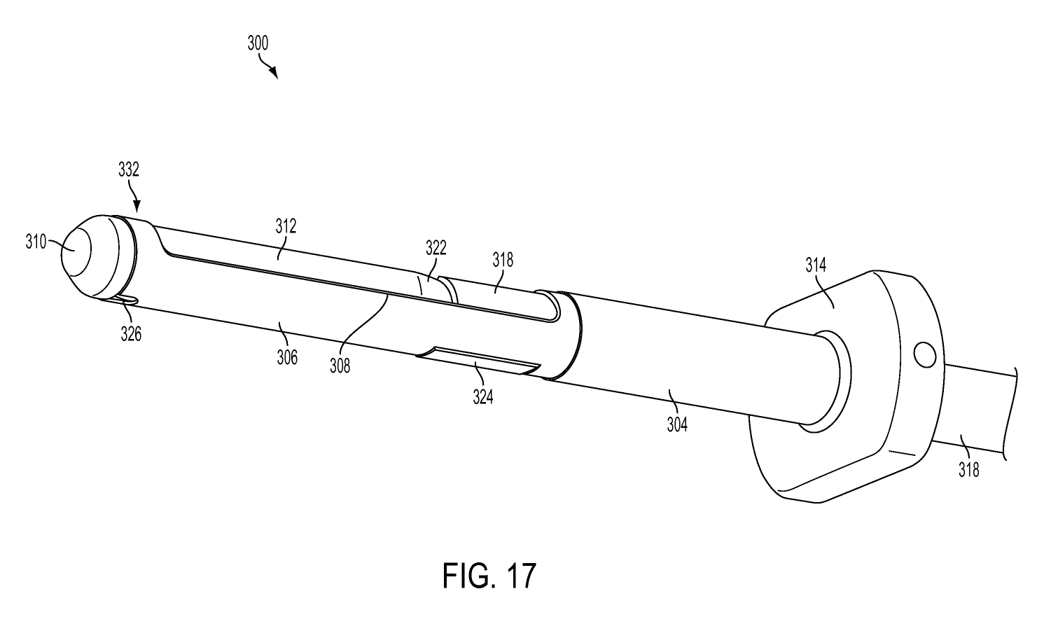

After the tip 310 and at least a portion of the shaft that includes the port 308 are inserted into a body cavity, the camera 312 may be released by moving grip 320 away from grip 314 (in the direction of arrow 330). This movement disengages the cavity 334 in the distal end of the rod 318 from the proximal end 322 of the camera 312. As shown in FIGS. 14-17, a spring 324, such as, for example, a leaf spring, may be included in the introducer 300 to push the proximal end 322 of the camera 312 out of the port 308. The spring 324 may be biased outwardly from the port 308, as shown in FIG. 14. When a surgeon inserts the camera 312 into the port 308, the spring 324 is moved to a nonbiased position, as shown in FIG. 17. Then, when the rod 318 is moved distally relative to the shaft 302 and engages the camera 312, as described above, the rod 318 maintains the spring 324 in the nonbiased position. After inserting the introducer 300 into a body cavity, the surgeon then pulls the rod 318 proximally relative to the shaft 302, disengaging the rod from the camera 312. Moving the rod 318 also frees the spring 324 to return to its biased position, and the spring 324 thereby pushes the proximal end 322 of the camera 312 out of the port 308. When the proximal end 322 of the camera 312 is pushed out of the port 308, as shown in FIGS. 14 and 15, an ECU may more easily extract the distal end 333 of the camera 312 from the distal cavity 332 than if the camera 312 were still completely within the port 308.

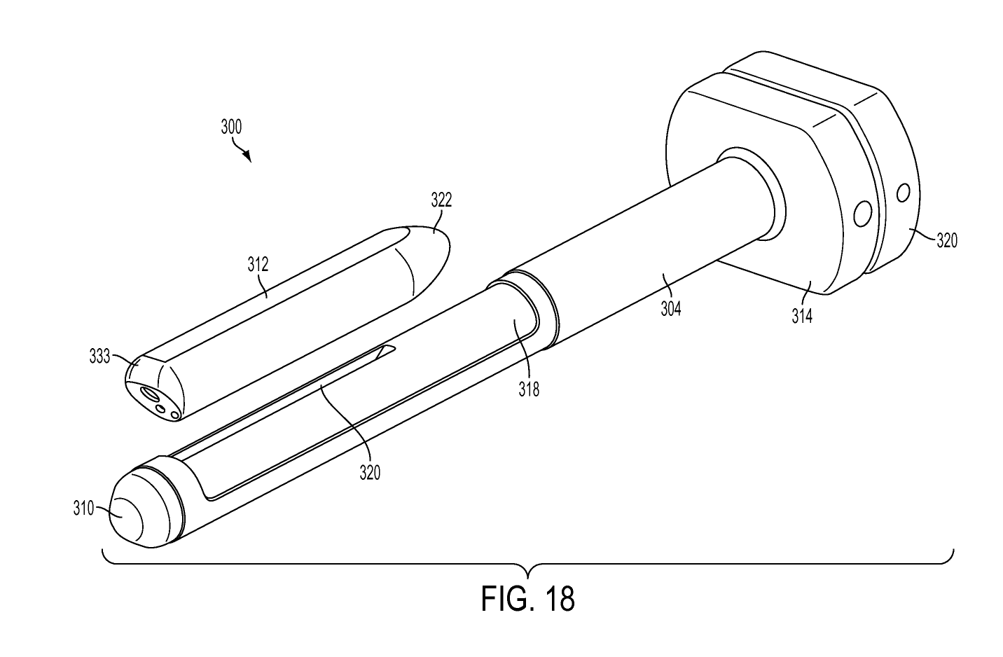

After the camera 312 is extracted from the introducer 300, the grip 320 may be moved towards grip 314 such that the rod 318 covers the cavity in the port 308 left behind by the camera 312. As shown in FIG. 18, grip 320 may be brought into contact with grip 314 such that the rod 318 fills the cavity in the port 308. Filling the cavity in the port 308 with the rod 318 may reduce the likelihood that the introducer 300 snags, scrapes, or otherwise damages tissue in the body cavity as the introducer 300 is extracted.

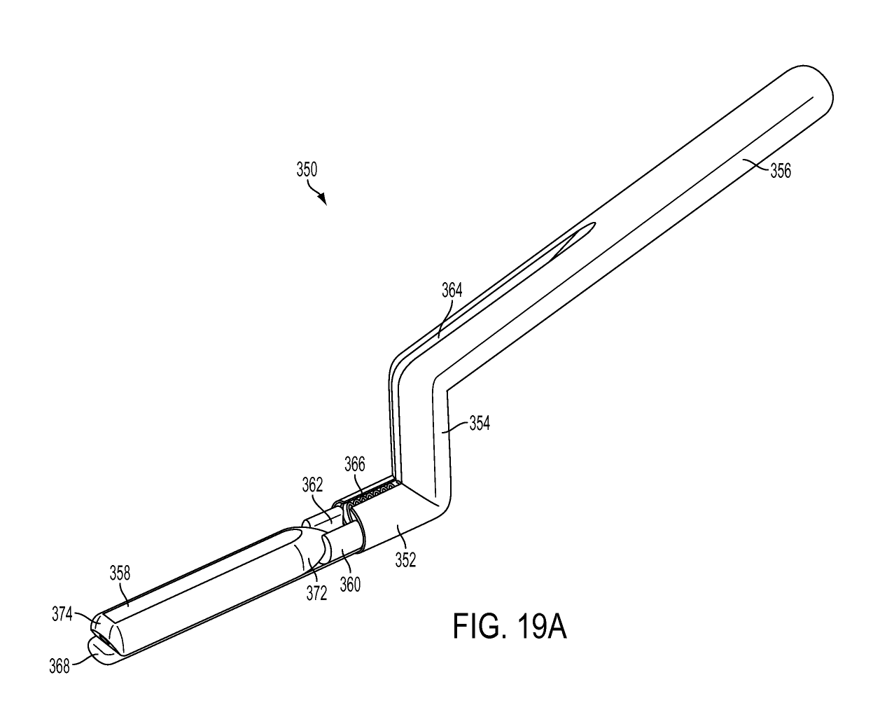

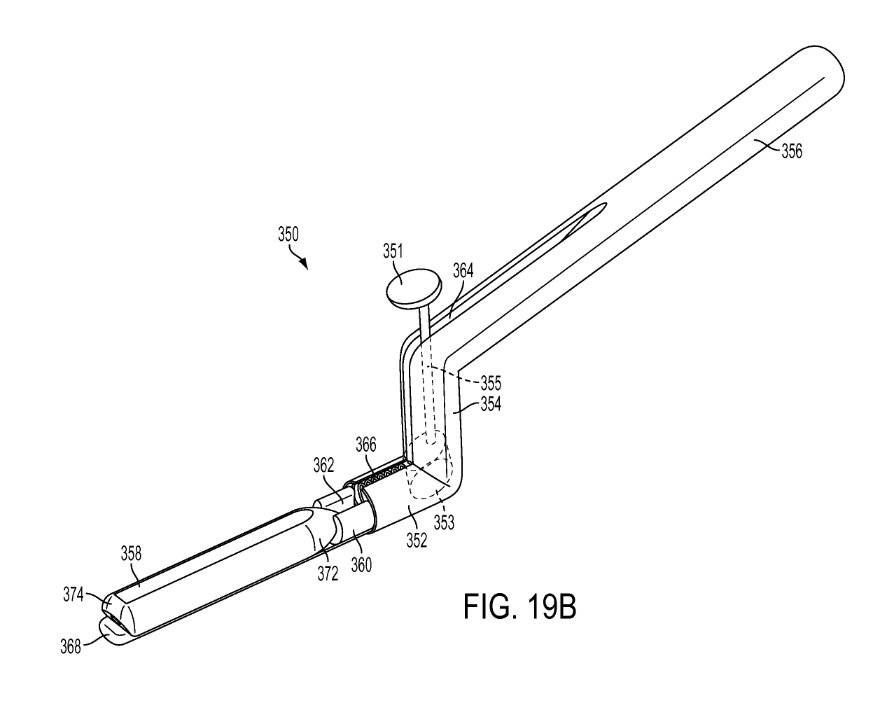

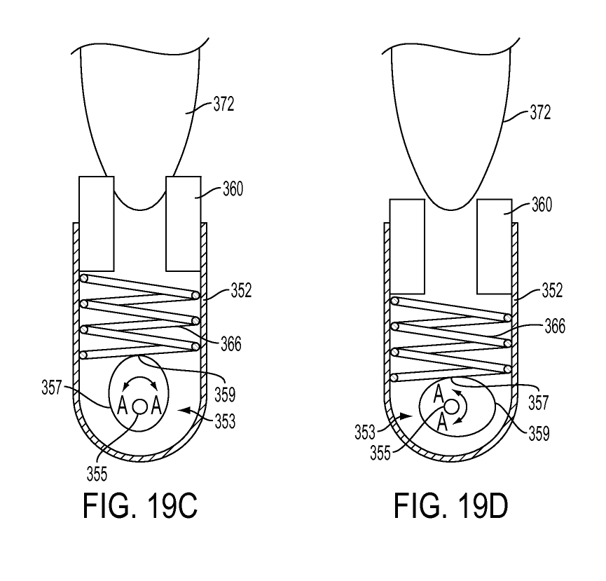

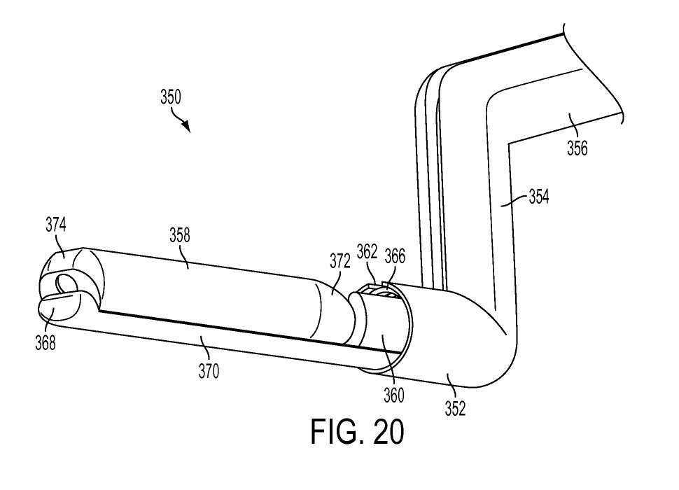

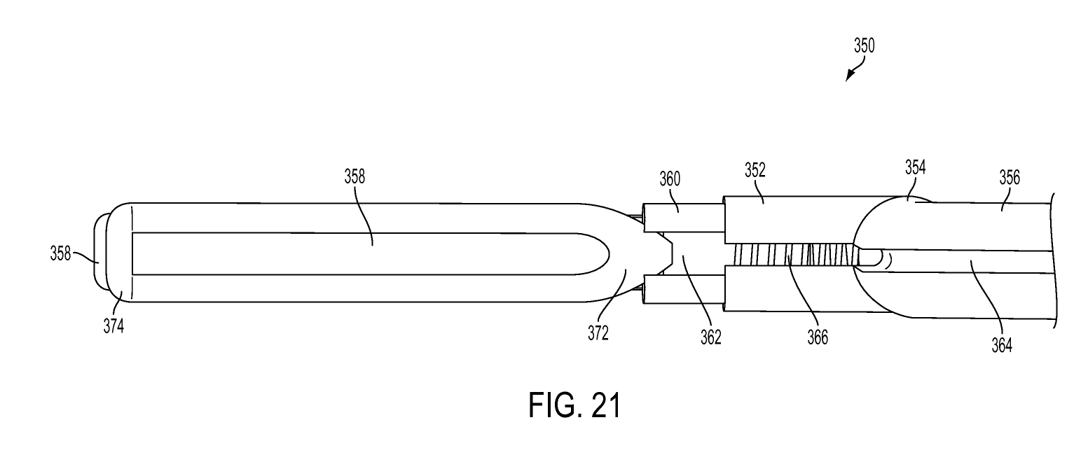

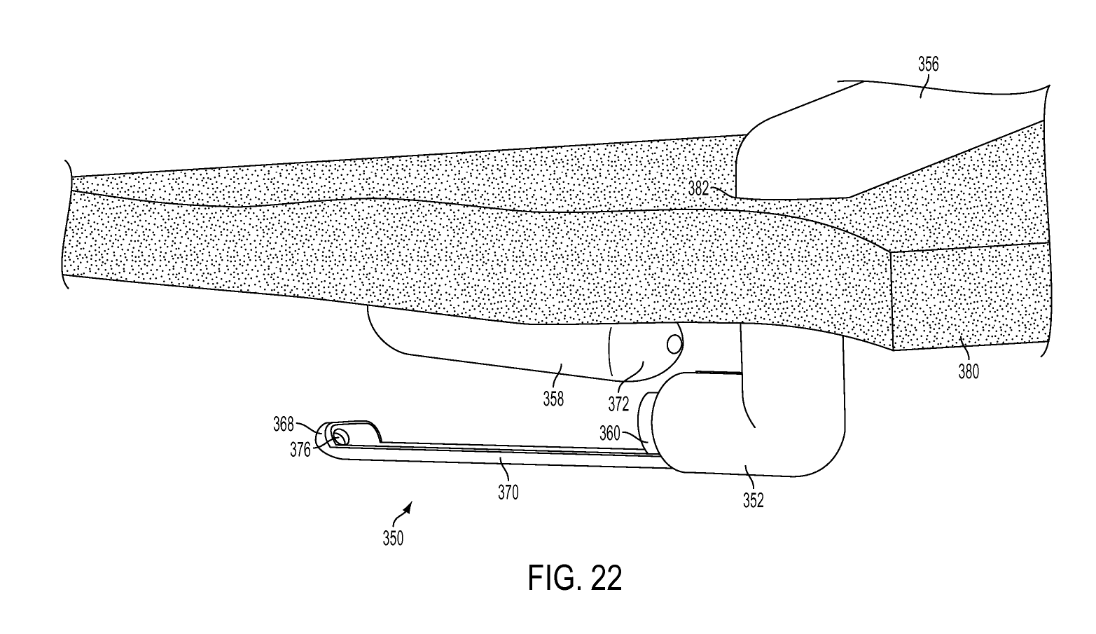

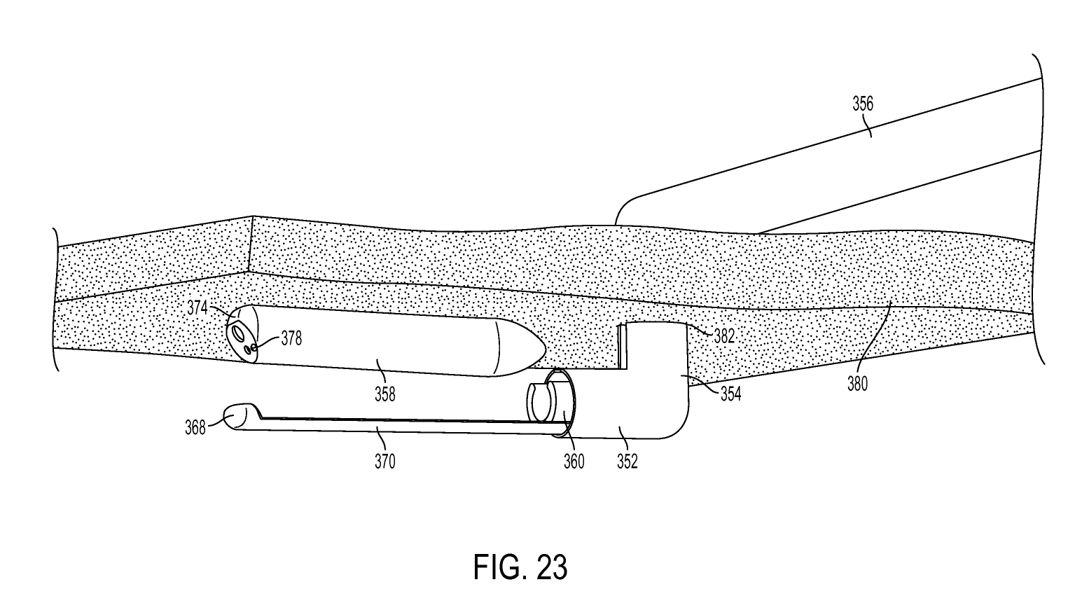

FIGS. 19A-23 show another embodiment of an introducer 350 for a camera 358. The introducer 350 comprises a shaft that includes a first portion 352, a second portion 354 attached to and at an angle relative to the first portion 352, and a third portion 356 attached to and at an angle relative to the second portion 354. The third portion 356 may be used as a handle that a surgeon may grip to manipulate the introducer 350. As shown in FIG. 20, a tray 370 and clip 368 may be attached to a distal end of the first portion 352 of the shaft. As shown in FIGS. 22 and 23, the first portion 352 of the shaft, and the tray 370 and the clip 368, can be inserted into a body cavity through an otomy 382 in tissue 380. The angles between portions 352 and 354 and portions 354 and 356 enable the tray 370 and clip 368 carrying the camera 358 to enter the body cavity at a shallow angle relative to the tissue 380. By introducing the camera 358 at a shallow angle, the camera 358 can more easily be oriented parallel to the tissue 380 for extraction of the camera 358.

The introducer 350 holds the camera 358 by clip 368 and retainer 360. The clip 368 may include an orifice 376 (shown in FIG. 22) that mates with a protrusion 378 (shown in FIG. 23) on the distal end 374 of the camera 358. Likewise, a retainer 360 engages a proximal end 372 of the camera. The retainer 360 may be coupled to a biasing mechanism, such as, for example, a spring 366. The spring 366 may bias the retainer 360 in a direction in which the retainer 360 engages the camera 358. In this configuration, after inserting the introducer 350 into a body cavity, a surgeon may operate a latch, switch, trigger, solenoid, or the like (not shown) to retract the retainer 360 away from the camera 358 to enable the camera 358 to be captured by an ECU (not shown) external to the body cavity as described above. The spring 366 may bias the retainer 360 in a direction in which the retainer 360 does not engage the camera 358. In this configuration, a latch or the like (not shown) may hold the retainer in a position in which it is engaged with the camera 358 (in other words, the latch holds the retainer 360 against the bias), and the surgeon may operate a switch or the like (not shown) to trigger the latch to release the retainer 360. The introducer 350 may comprise a slot 364 configured to accommodate a tether (not shown) of the camera 358.

FIGS. 19B-19D show an embodiment of an engagement member 353 for selectively pressing the spring 366 and the retainer 362 against the camera 358 to retain the camera 358. The engagement member 353, as shown in FIGS. 19C and 19D, may include an eccentric cam defining a small-radius surface 357, a large-radius surface 359, and continuously varying radii therebetween. The eccentric cam 353 rotates about a shaft 355 in the direction of arrow “A,” and the shaft 355 is connected to a knob 351 that a surgeon may use to turn the shaft 355 and the cam 353. In the position shown in FIG. 19C, the large-radius surface 359 of the cam 353 pushes the spring 366 and the retainer 360 against the proximal end 372 of the camera. When rotated to the position shown in FIG. 19D, the small-radius surface 357 of the cam 353 is now in contact with the spring 366, and the cam 353 enables the spring 366 and the retainer 372 to move away from the proximal end 372 of the camera, freeing the camera from the introducer 350. Rather than enabling the spring 366 and retainer 360 to back away from the camera, the movement of the cam 353 from the position shown in FIG. 19C to FIG. 19D may instead reduce compression forces on the spring 366 such that an ECU may pull the proximal end 372 of the camera from the retainer 360.

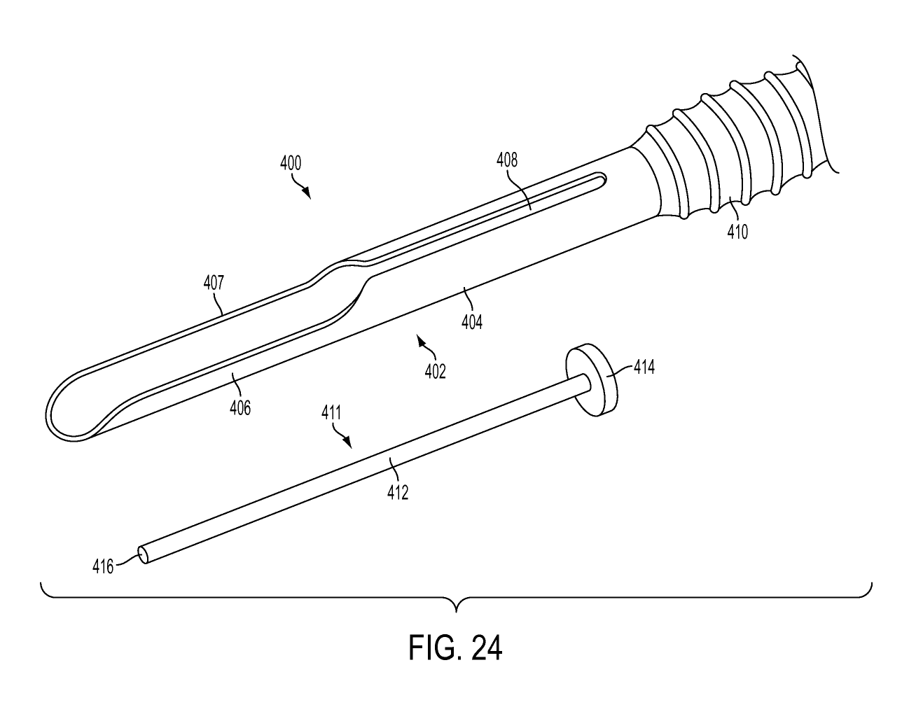

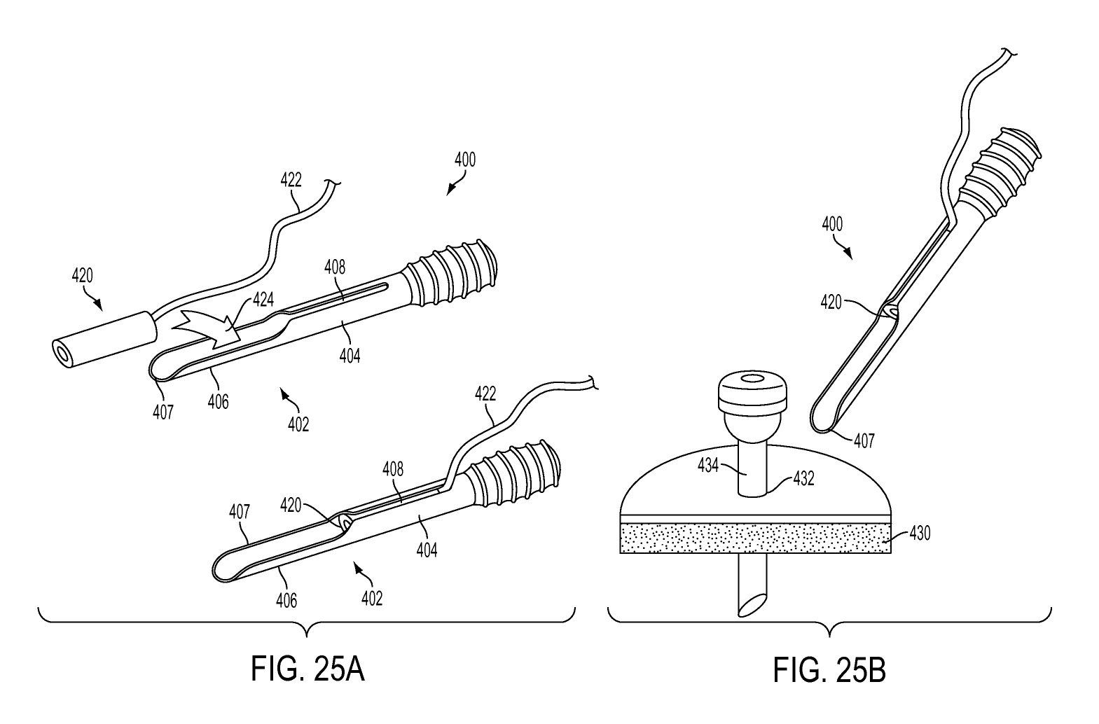

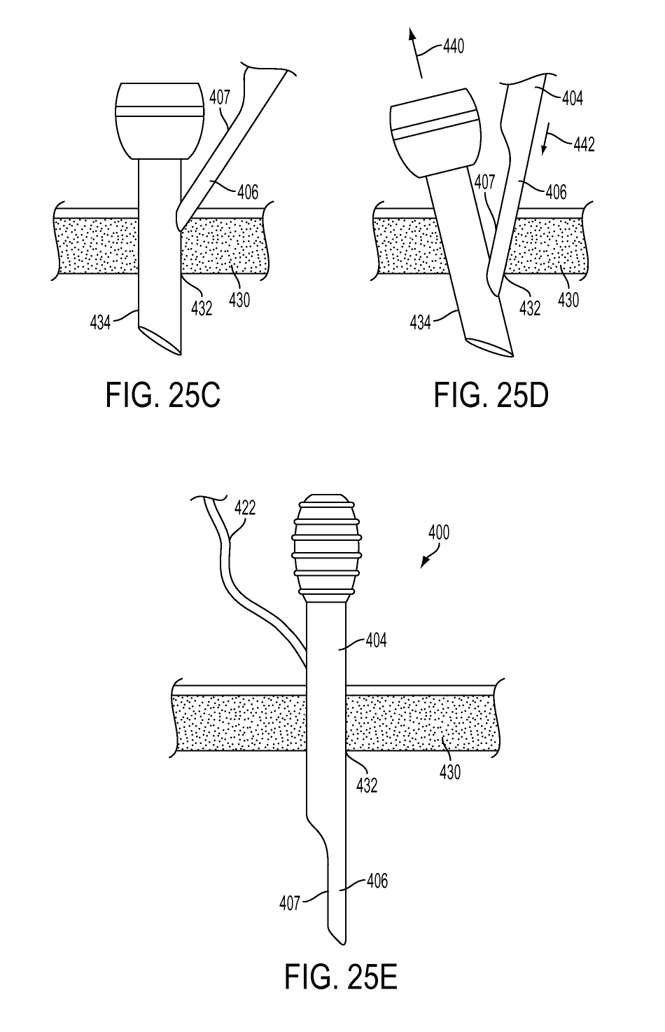

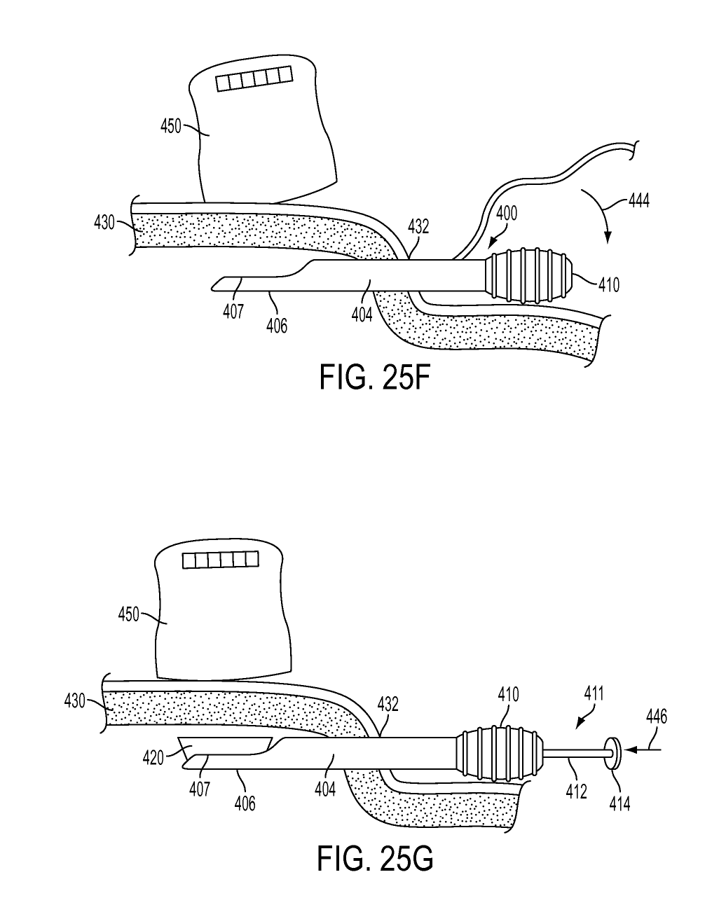

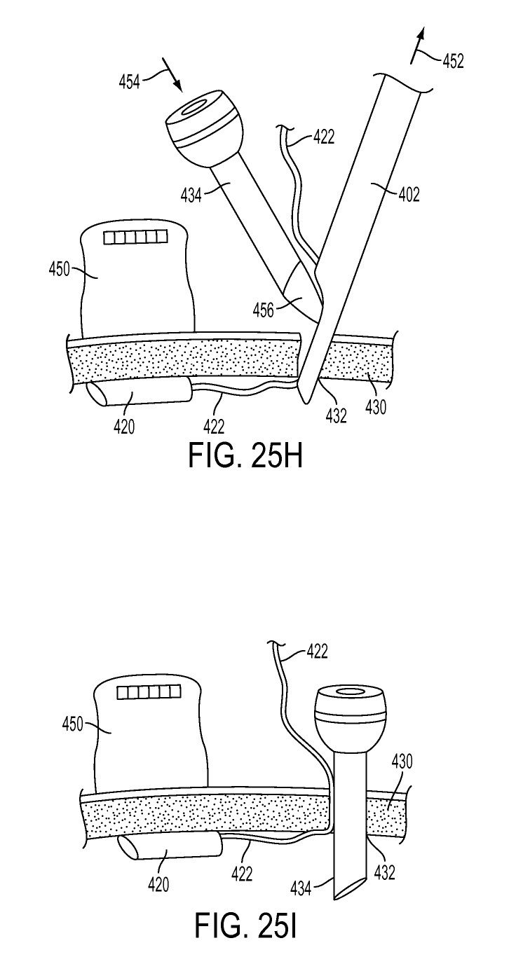

FIG. 24 shows another embodiment of an introducer 400 for an internal magnetic camera (not shown). The introducer 400 may include a shaft 402 and a handle 410, wherein the shaft 402 includes a distal shaft portion 406 and a proximal shaft portion 404. The shaft 402 may be generally hollow and the handle 410 also may include a passage (not shown) therethrough that is in communication with the hollow shaft 402. The hollow shaft 402 may have a generally circular cross-section, which transitions into a generally arcuate open region 407. The hollow shaft 402 may be configured to receive a camera 420 (shown in FIGS. 25A-I). The introducer 400 may use a ram rod 411 to remove the camera from the introducer 400. The ram rod 411 may include a shaft 412 that comprises a distal end 416 and a grip 414 at an opposite end. The distal end 416 of the shaft 412 may be inserted into the passage (not shown) in the handle 410 and into the shaft 402 of the introducer 400 to push a camera out, as described below.

FIGS. 25A-I show steps that may be followed to use the introducer 400. As shown in FIG. 25A, a camera 420 is inserted into the shaft 402 by placing the camera 420 on the open region 407 of the distal shaft portion 406 and then sliding the camera 420 relative to the shaft 402 in the direction of arrow 424 so that the camera is captured in the proximal shaft portion 404. A tether 422 of the camera 420 may be received by a slot 408 in the shaft 402. After the camera 420 is loaded into the proximal shaft portion 404, the introducer 400 is oriented relative to a trocar shaft 434 in place in an otomy 432 in tissue 430 of a body cavity. The shaft 402 of the introducer 400 is oriented so that an interior surface of the open region 407 aligns with an exterior surface of the trocar shaft 434. Preferably, the interior surface of the open region 407 includes an arcuate configuration, the radial dimensions of which correspond to, but are slightly greater than the radial dimensions of the trocar shaft 434. As shown in FIG. 25C, the distal shaft portion 406 contacts the trocar shaft 434, and the outer surface of the trocar shaft 434 aligns with the inner surface of the open region 407 such that the combined trocar-shaft 434 and open region 407 has a cross-sectional area only slightly larger than the trocar shaft 434 above. Then, as shown in FIG. 25D, the introducer 400 is moved in the direction of arrow 442 so that the distal shaft portion 406 enters the otomy 432 along the side of the trocar shaft 434. The match of the inner surface of the open region 407 to the trocar shaft 434 minimizes any stretching or further opening of the otomy 432 when both the trocar shaft 434 and the distal shaft portion 406 are positioned in the otomy 432. As the distal shaft portion 406 is inserted into the otomy 432, the trocar shaft 434 may be removed from the otomy 432 in the direction indicated by arrow 440. Eventually, the trocar shaft 434 may be completely removed from the otomy 432, leaving the shaft 402 of the introducer 400 inserted in the otomy 432 (as shown in FIG. 25E).

With the introducer 400 inserted in the otomy 432, the shaft 402 may be rotated about the otomy 432 by moving the handle 410 in the direction of arrow 444 (shown in FIG. 25F). This rotation of the shaft 402 brings the distal shaft portion 406 approximately parallel to the tissue 430. An ECU 450 can be positioned external to the tissue 430 and opposite the distal shaft portion 406. The ram rod 411 then may be inserted into the passageway (not shown) in the handle 410 and pushed in the direction of arrow 446 to move the camera 420 relative to the shaft 402 from the proximal shaft portion 404 to the open region 407 in the distal shaft portion 406. The ECU 450 then may be energized to pick up the camera 420 from the shaft 402.

After the camera 420 is released from the introducer 400, the introducer 400 may be withdrawn from the otomy 432 as shown in FIGS. 25H and 25I. As shown in FIG. 25H, the shaft 402 of the introducer 400 is pulled out in direction indicated by arrow 452 as the trocar shaft 434 is inserted into the otomy 432 in the direction of arrow 454. The trocar shaft 434 again matches the contour of the inner surface of open region 407 to minimize stretching or opening of the otomy 432. The trocar shaft 434 may include an obturator tip 456 at its distal end, as shown in FIG. 25H. After the trocar shaft 434 is re-introduced into the otomy 432, the introducer may be completely removed, as shown in FIG. 25I, leaving the camera 420 inside the body cavity and the tether 422 passing through the otomy 432 and next to the trocar shaft 434.

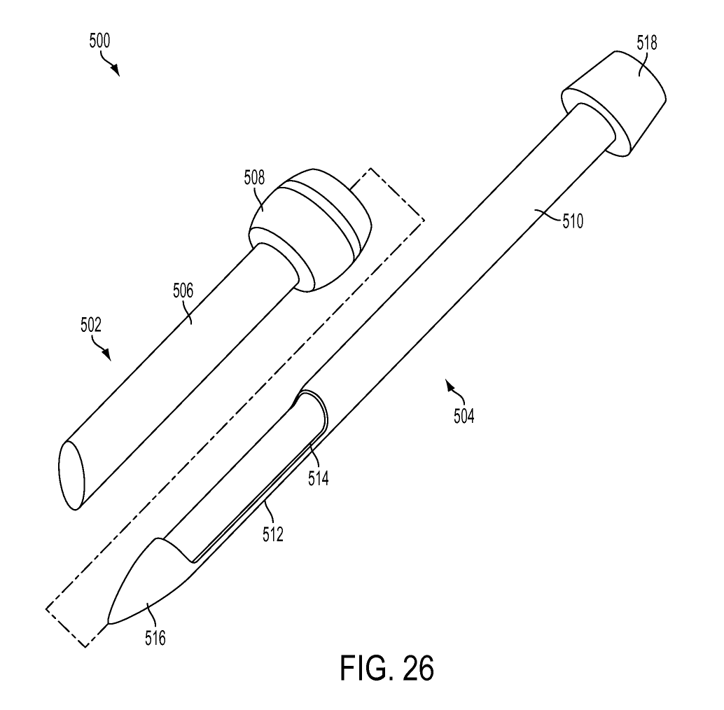

FIG. 26 shows another embodiment of an introducer 500 for an internal magnetic camera that comprises a first shaft 502 and a second shaft 504. The first shaft 502 may include a hollow tube 506 and a handle 508 that may also be hollow or may have a passage therethrough. The second shaft 504 may include a proximal shaft portion 510 and a distal shaft portion 512. The distal shaft portion 512 may include a port 514 on a side configured to receive a camera (not shown). The second shaft 504 may further comprise a tip 516, such as, for example, a sharp piercing tip that is typically used with a trocar, attached to the distal shaft portion 512, and a handle 518 attached to the proximal shaft portion 510. The first shaft 502 may be positioned over the second shaft 504 such that the first shaft 502 and the second shaft 504 are coaxial. The first shaft 502 may slide relative to the second shaft 504. The shaft 504 may be inserted through the hollow or passage in handle 508, with the tip 516 as the leading end and passing through shaft 502. The handle 518 prevents shaft 506 and handle 508 from being pulled too far proximally and off of shaft 504.

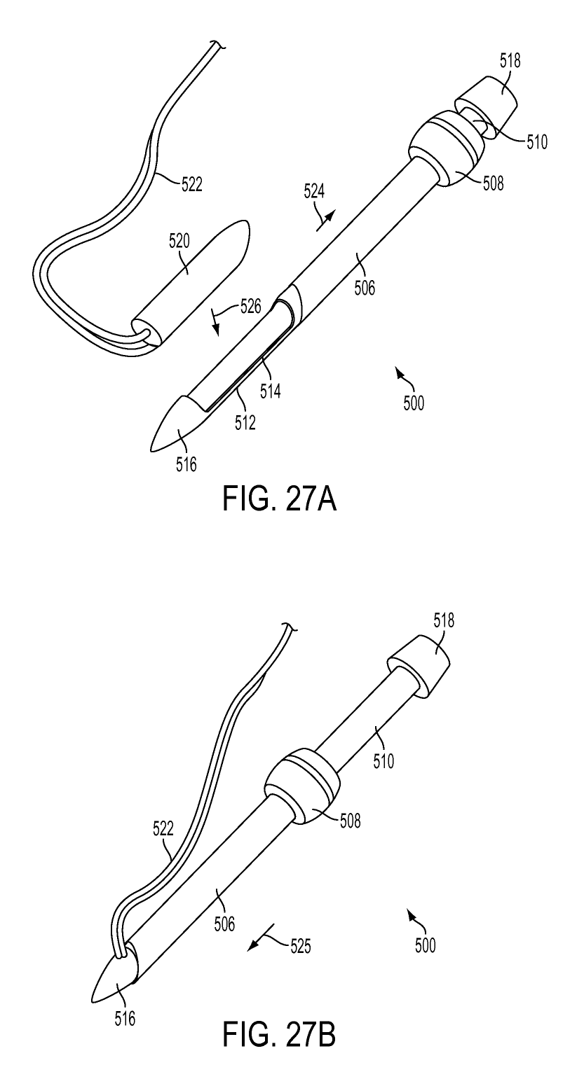

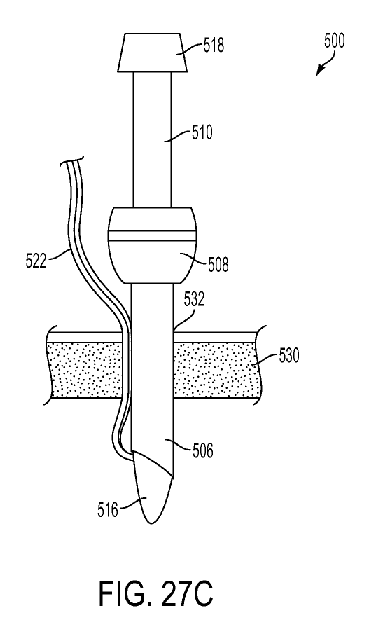

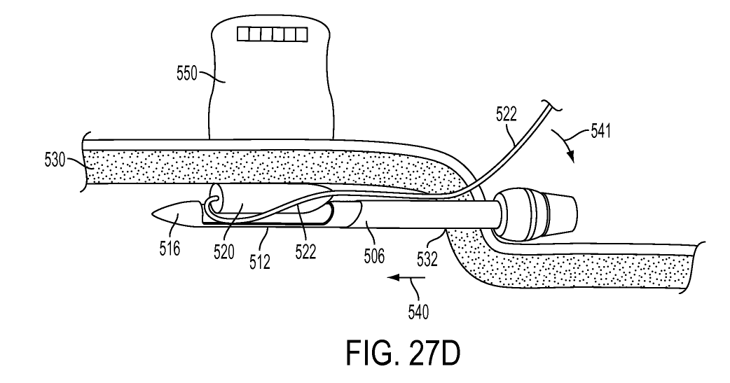

FIGS. 27A-D illustrate a method for using the introducer 500 of FIG. 26. As shown in FIG. 27A, the hollow tube 506 of the first shaft 502 may be slid relative to the second shaft 504 in the direction of arrow 524 until the port 514 is accessible. Then, a magnetic camera 520 may be loaded into the port 514 as indicated by arrow 526. In this embodiment, the camera 520 may loaded in a reverse direction, i.e., with its tether 522 closest to the distal tip 516 of the second shaft 504. After the camera 520 is loaded into the port 512, the first shaft 502 is moved relative to the second shaft 504 in the direction of arrow 525 (shown in FIG. 27B) to cover the port 512 and the camera 520 contained therein. The tether 522 of the camera 520 exits the port 512 from a distal end of the hollow tube 506 of the first shaft 502.

With the camera 520 loaded in the port 514 and covered by the first shaft 502, the introducer 500 may be inserted into an incision or otomy 532 in animal tissue 530, such as, for example, an otomy through an abdominal wall, as shown in FIG. 27C. As described above, the tip 516 of the introducer 500 may be sharp and may comprise a cutting edge such that the tip 516 may create the otomy 532. The tip 516 of the introducer may be blunt and may guide the introducer 500 through an already-created otomy 532. The tether 522 of the camera 520 follows the tip 516 through the otomy 532. The introducer 500 may be inserted into the otomy 532 far enough that a substantial portion of the hollow tube 506 of the first shaft 502 is passed into the otomy 532, as shown in FIG. 27C. Then, the introducer 500 is rotated in the direction indicated by arrow 541 in FIG. 27D. After rotation, the second shaft 504 is moved relative to the first shaft 502 in the direction indicated by arrow 540 in FIG. 27D. Preferably, when the second shaft 504 is moved relative to the first shaft 502, the first shaft 502 is stationary relative to the otomy 532 in which it is inserted. The relative motion of the first shaft 502 and the second shaft 504 shown in FIG. 27D exposes the port 514 and the camera 520 contained therein. An ECU 550 outside of the body tissue 530 then may be energized, which will extract the camera 520 from the port 514. After the camera 520 is extracted from the port 514, the introducer may be removed from the otomy 532. Optionally, the first shaft 502 may be left in place in the otomy 532 and used as a guide for other endoscopic instruments, wherein only the second shaft 504 is withdrawn from the otomy 532 completely. The introducer 500 may be reinserted into the body cavity to remove the camera, as described above with regard to other embodiments of the introducer.

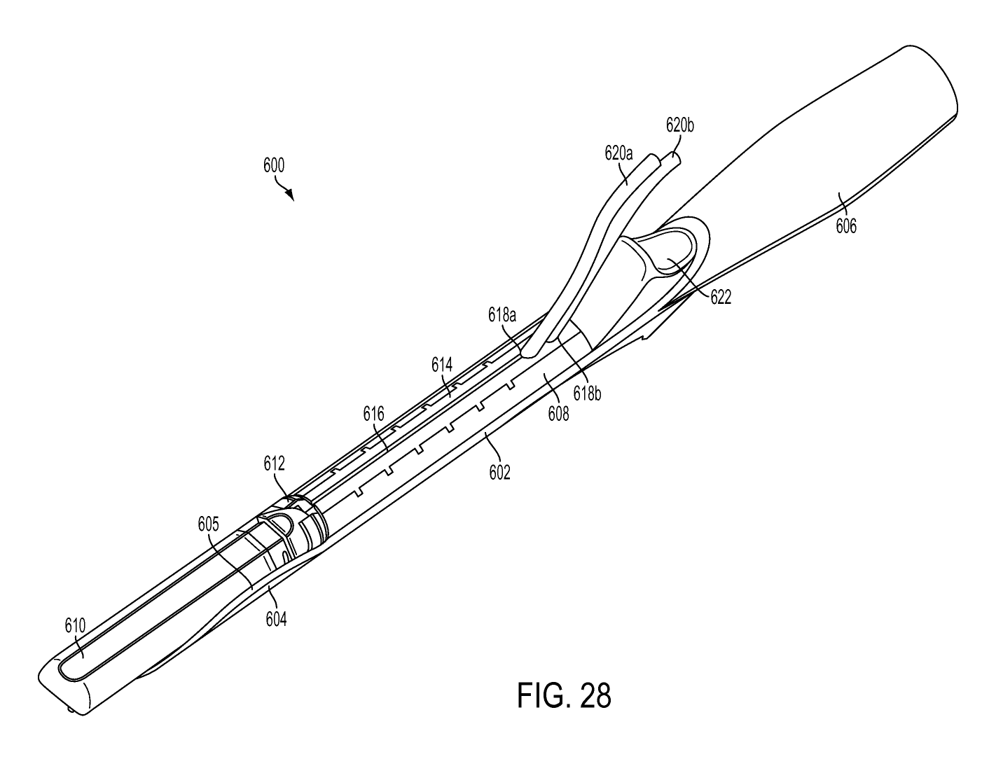

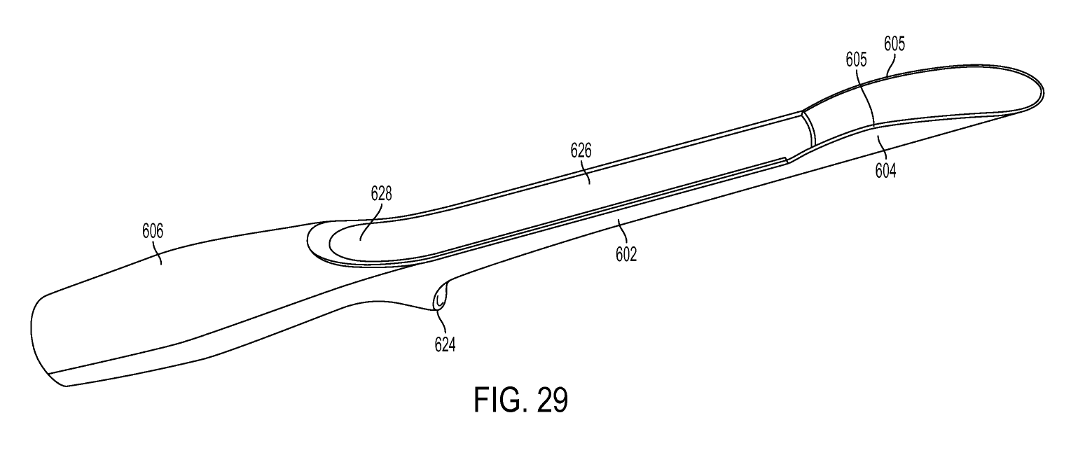

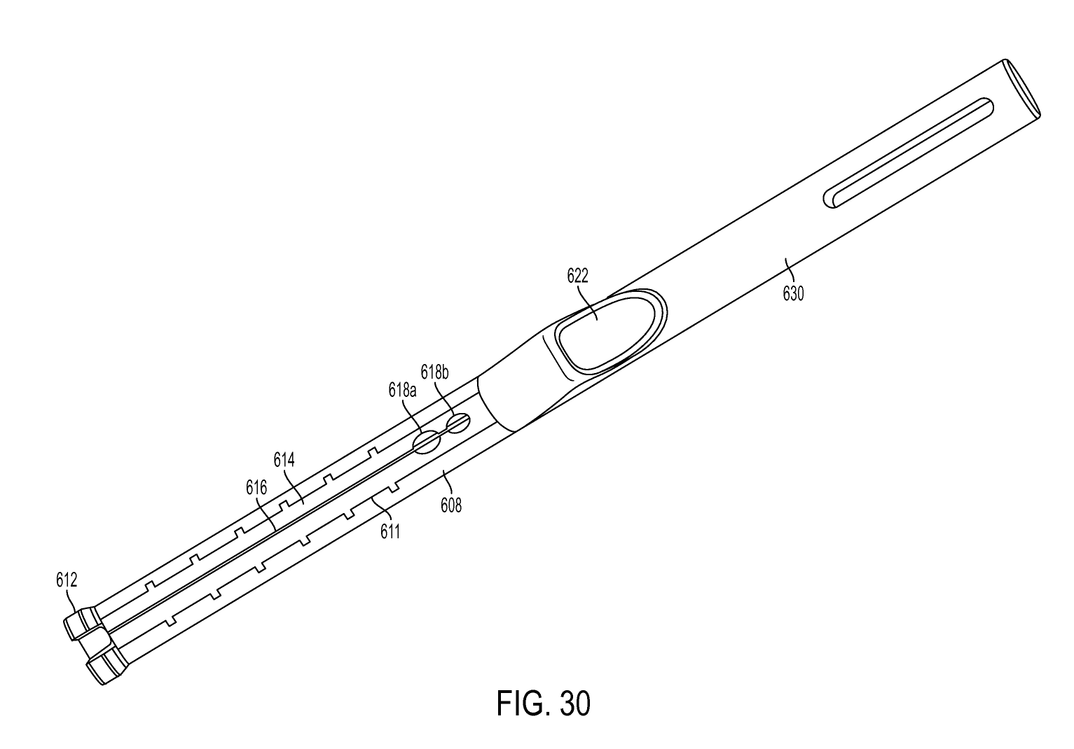

FIGS. 28-32 show another embodiment of an introducer 600. The introducer 600 includes an arcuate, such as, for example, a shoe-horn-shaped, distal channel 604 into which a camera 610 is placed, and the camera 610 and arcuate distal channel 604 are inserted into an otomy or incision (not shown) in tissue. The introducer 600 comprises two main components: a body (shown in FIG. 29) and a plunger (shown in FIG. 30).

The body may be made of any biocompatible material, such as, for example, plastic or metal, and includes a handle 606 and a proximal channel 602 and the arcuate distal channel 604. The handle 606 may be hollow. In various embodiments, the proximal channel 602 may have a semi-circular or arced contour 626. The arcuate distal channel 604 may be connected to the proximal channel 602 as a continuous integrated portion or welded or molded onto the end. The arcuate distal channel 604 generally continues the semi-circular contour 626 of the proximal channel 602, except that the arcuate distal channel 604 may include wings 605 that extend upwardly, in a generally u-shaped configuration, or may curve inwardly towards each other to continue the arc of the cross-section beyond a semi-circle and closer to a full circle. The distal end of the arcuate distal channel 604 may flare outwardly to provide a wider channel. For example, the distal channel 604 may transition into an arc that may have greater radial dimensions than the remainder of the arcuate distal channel 604.

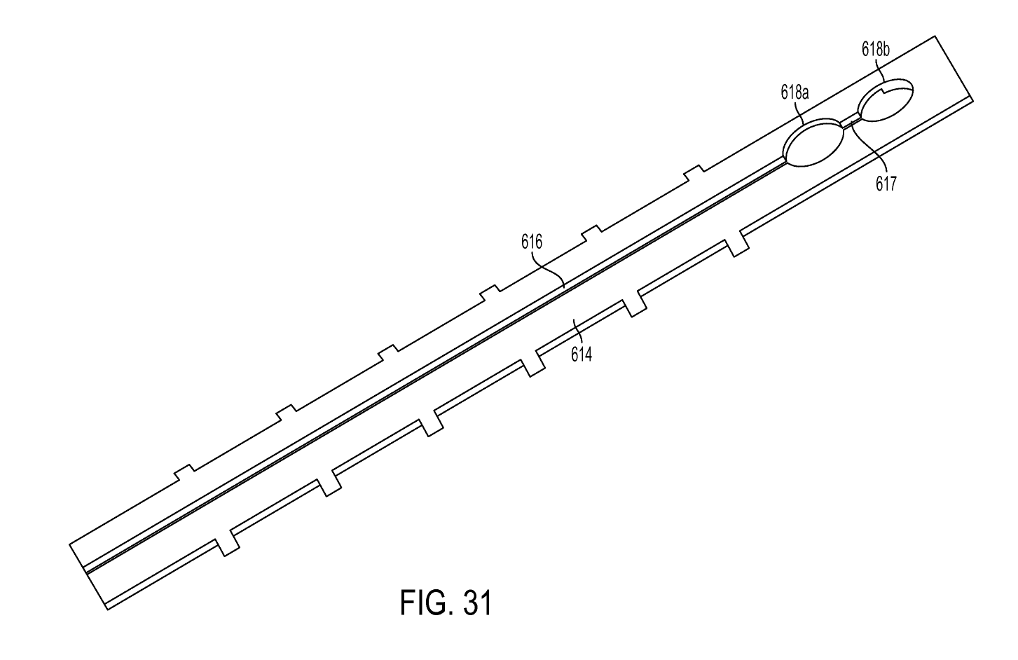

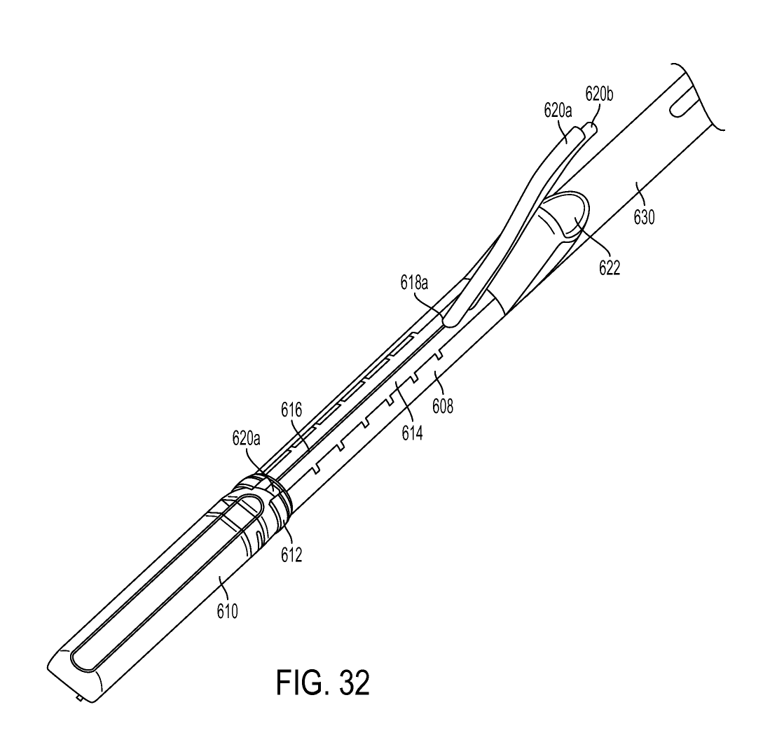

The plunger also typically is made of the same biocompatible materials as the body, such as a plastic, such as, for example, nylon or polycarbonate, and may fit in the channel 626 of the body and may slide relative to the body. The plunger may include a proximal end 630 that fits into and slides relative to the hollow handle 606. A distal end of the plunger includes a receiver 612 that receives a distal end of the camera 610. The plunger may include a longitudinal slot or channel 611 that receives tethers 620a and 620b from the camera 610. The slot or channel 611 may include a seal 614 therein. The seal 614 may be made of silicone, rubber, neoprene, or any other compliant material. The seal 614 may be attached to the slot 611 by any means, such as, for example, overmolding, comolding, adhesive, or fasteners. The seal 614 (shown by itself in FIG. 31) may include apertures 618a and 618b through which the tethers 620a and 620b pass when the camera 610 is loaded in the introducer 600. The apertures 618a and 618b preferably are sized to snugly fit around respective tethers 620a and 620b to minimize leakage of insufflation gases. The seal also may include a first longitudinal slit 616 from its distal end to the aperture 618a and a second longitudinal slit 617 between the apertures 618a and 618b. FIG. 32 shows the camera 610 received by the receiver 612 of the plunger. The tethers 620a and 620b pass through the channel 611 and out of the apertures 618a and 618b. The camera 610 may be held loosely in the shoe-horn-shaped distal channel 604 by the wings 605.

With the camera 610 loaded in the introducer 600, a surgeon may grasp the handle 606, keeping a finger or a portion of his hand in contact with the camera 610. As described above, the camera 610 may be loosely retained by the wings 605, and the surgeon's finger (or portion of the hand) supporting the camera 610 may be necessary to maintain the camera 610 in the receiver 612. While maintaining his grip on the handle 606 and the camera 610, the surgeon may push the shoe-horn-shaped distal channel 604 and the camera 610 into a body cavity, such as, for example, an abdominal cavity through an otomy. After the camera 610 has at least partially penetrated the otomy, the surgeon may remove his finger from the camera 610 as the tissue of the otomy and of the body cavity may hold the camera 604 in place on the shoe-horn-shaped distal channel 604. After the camera 610 has fully penetrated the otomy, the surgeon may torque the introducer 600 (as described above with respect to other embodiments) to bring the camera 610 proximate and parallel to the tissue of the body cavity. The surgeon then pushes the plunger distally relative to the body to push the camera 610 out of the introducer 600. The surgeon may push against tab 622 on the plunger and tab 624 on the body to push the plunger relative to the body. As the camera 610 is removed from the introducer 600, the tethers 620a and 620b are pulled out of the apertures 618a and 618b and through the slots 616 and 617 in the seal 614. The introducer 600 then can be withdrawn from the otomy, leaving the camera 610 within the body cavity and the tethers 620a and 620b passing through the otomy.

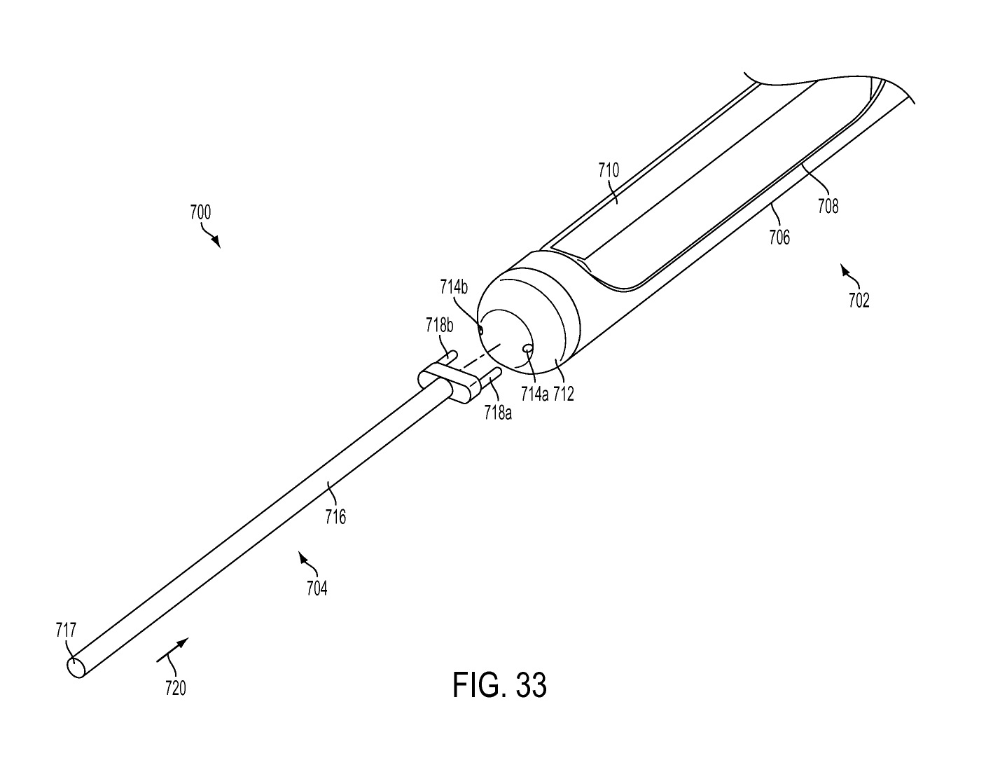

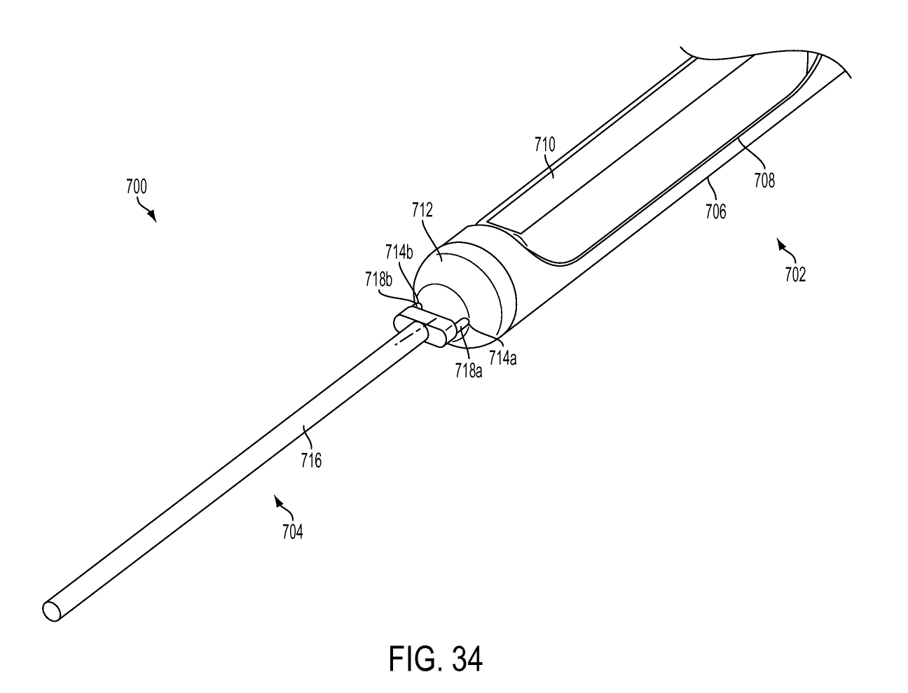

FIGS. 33 and 34 show another embodiment of an introducer 700 comprising a heating element 704 that preheats an internal magnetic camera 710 prior to insertion into a body cavity. Operating rooms generally are cold, such as, for example, colder than the temperature of a human body, at least for the reason of discouraging growth of bacteria and other organisms. Consequently, operating instruments, such as the introducers and internal magnetic cameras discussed above, that may be stored in the operating room also may be cold. If a cold camera is introduced into a warm, relatively humid body cavity, then the camera's lens may be obscured by condensation that may form. Various embodiments of the introducers described herein, such as introducer 702, shown in FIG. 33, may have a tip 712 that may include ports 714a and 714b to receive a heating element 704. The heating element 704 may include a shaft 716 coupled to a heat source (not shown) at its distal end 717 and plugs 718a and 718b at its proximal end. The heating element comprises a material that will transmit heat from the heat source to the plugs 718a and 718b. By moving the heating element in the direction indicated by arrow 720, the plugs 718a and 718b can be plugged into respective ports 714a and 714b in the tip 712 of the introducer as shown in FIG. 34. Heat from the heating element 704 can be transmitted to the tip 712 through the plugs 718a and 718b that are plugged into respective ports 714a and 714b. This heat is then transferred to the camera 710, in a port 708 of the shaft 706 of the introducer 700. Preferably, the camera 710 is heated to no more than 110° Fahrenheit (approximately 43° Celsius) prior to insertion into the body. Embodiments of the heating element may comprise a pouch (not shown) that may be placed on the camera 710 in the introducer 700. The pouch may comprise chemicals that react exothermally, heating the camera 710 on which it is placed.

Endoscopic minimally invasive surgical and diagnostic medical procedures are used to evaluate and treat internal organs by inserting a small tube into the body. The endoscope may have a rigid or a flexible tube. A flexible endoscope may be introduced either through a natural body opening (e.g., mouth, nose, anus, and/or vagina) or via a trocar through a relatively small—keyhole—incision incisions (usually 0.5-2.5 cm). The endoscope can be used to observe surface conditions of internal organs, including abnormal or diseased tissue such as lesions and other surface conditions and capture images for visual inspection and photography. The endoscope may be adapted and configured with working channels for introducing medical instruments to the treatment region for taking biopsies, retrieving foreign objects, and/or performing surgical procedures.

All materials used that are in contact with a patient are preferably made of biocompatible materials.

Preferably, the various embodiments of the devices described herein will be processed before surgery. First, a new or used instrument is obtained and if necessary cleaned. The instrument can then be sterilized. In one sterilization technique, the instrument is placed in a closed and sealed container, such as a plastic or TYVEK® bag. The container and instrument are then placed in a field of radiation that can penetrate the container, such as gamma radiation, x-rays, or high-energy electrons. The radiation kills bacteria on the instrument and in the container. The sterilized instrument can then be stored in the sterile container. The sealed container keeps the instrument sterile until it is opened in the medical facility. Other sterilization techniques can be done by any number of ways known to those skilled in the art including beta or gamma radiation, ethylene oxide, and/or steam.

Although the various embodiments of the devices have been described herein in connection with certain disclosed embodiments, many modifications and variations to those embodiments may be implemented. For example, different types of end effectors may be employed. Also, where materials are disclosed for certain components, other materials may be used. The foregoing description and following claims are intended to cover all such modification and variations.

Any patent, patent application, publication, or other disclosure material, in whole or in part, that is said to be incorporated by reference herein is incorporated herein only to the extent that the incorporated materials does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

1. An introducer for an internal magnetic camera, comprising:
a shaft comprising a distal end and a proximal end, at least a portion of the shaft being hollow, the hollow portion having an inner diameter configured to carry an internal magnetic camera therein, the hollow portion further comprising a port through which the internal magnetic camera may be inserted into the shaft or removed from the shaft; and
a retainer arranged to selectively maintain the internal camera in the hollow portion.

2. The introducer of claim 1, wherein the retainer comprises a magnetic material in the hollow portion, the magnetic material providing a magnetic coupling force between the magnetic material and a magnet in the internal magnetic camera that is weaker than a magnetic coupling between an external control unit (ECU) and the magnet in the internal magnetic camera.

3. The introducer of claim 1, wherein the retainer comprises a clip configured to mate with a surface of the internal magnetic camera; and
the retainer further comprises an engagement member moveable between a first position and a second position, wherein in the first position the engagement member is positioned relative to the clip to capture the internal magnetic camera, and wherein in the second position the engagement member is positioned relative to the clip such that the internal magnetic camera is not captured.

4. The introducer of claim 3, wherein the clip further comprises a mating surface that is configured to engage a corresponding mating surface on the internal magnetic camera, wherein the mating surfaces, when engaged, orient the internal magnetic camera relative to the clip.

5. The introducer of claim 3, further comprising a spring coupled to the engagement member structured to bias the engagement member in the first position.

6. The introducer of claim 3, further comprising a spring coupled to the engagement member structured to bias the engagement member in the second position.

7. The introducer of claim 3, further comprising a handle coupled to the shaft; and
further comprising a plunger having a proximal end and a distal end, the distal end of the plunger being coupled to the engagement member, the proximal end of the plunger being accessible on the handle, and wherein movement of the plunger from a first position to a second position moves the engagement member from the engagement member's first position to the engagement member's second position.

8. The introducer of claim 1, wherein the retainer comprises a first portion that defines an opening configured to receive a protrusion on the internal magnetic camera, and the retainer further comprises a second portion that is movable between a first position and a second position, wherein in the first position magnetic the first and second portions of the retainer are positioned to retain the internal magnetic camera, and wherein in the second position the first and second portions are positioned such that the internal magnetic camera is not retained.

9. The introducer of claim 8, wherein the retainer further comprises a mating surface that is configured to engage a corresponding mating surface on the internal magnetic camera, wherein the mating surfaces, when engaged, orient the internal magnetic camera relative to the retainer.

10. The introducer of claim 8, further comprising a spring coupled to the second portion of the retainer structured to bias the movable portion of the retainer in the first position.

11. The introducer of claim 8, further comprising a spring coupled to the second portion of the retainer structured to bias the movable portion of the retainer in the second position.

12. The introducer of claim 8, further comprising a handle coupled to the shaft; and
further comprising a plunger having a proximal end and a distal end, the distal end of the plunger being coupled to the second portion of the retainer, the proximal end of the plunger being accessible on the handle, and wherein movement of the plunger from a first position to a second position moves the second portion of the retainer from the second portion's first position to the second portion's second position.

13. The introducer of claim 1, further comprising a longitudinal slot in the shaft configured to accommodate a tether attached to an internal magnetic camera.

14. The introducer of claim 1, wherein the port comprises an opening at the distal end of the hollow portion.

15. The introducer of claim 1, wherein the port comprises an opening in a wall of the shaft along the hollow portion.

16. The introducer of claim 1, wherein the shaft defines a passage for receiving a trocar shaft, the trocar shaft further comprising a tip.

17. The introducer of claim 16, wherein the tip comprises a transparent plastic material.

18. The introducer of claim 16, wherein the tip comprises an opaque material and an aperture through which the internal magnetic camera may receive an image.

19. The introducer of claim 16, wherein the tip comprises a cutting surface.

20. The introducer of claim 1, further comprising an ejection member that is configured to eject the internal magnetic camera, wherein the ejection member is restrained from ejecting the internal magnetic camera when the retainer maintains the camera in the hollow portion, and wherein the ejection member ejects the internal magnetic camera when the retainer does not selectively maintain the camera in the hollow shaft.

21. The introducer of claim 20, wherein the ejection member comprises a spring.

22. The introducer of claim 1, wherein the shaft includes a proximal section and a distal section, wherein the proximal and distal sections are at an angle relative to each other.

23. The introducer of claim 1, wherein the distal end of the shaft comprises an open region configured to conformingly mate to an exterior surface of a trocar, and wherein the open region is further configured to slide relative to the exterior surface of the trocar.

24. The introducer of claim 23, wherein the hollow shaft further comprises an open end at its proximal end; and
further comprising a ram rod configured to slide relative to the hollow shaft through the open end at the proximal end of the hollow shaft, wherein distal movement of the ram rod relative to the hollow shaft displaces the internal magnetic camera from the hollow shaft.

25. An introducer for an internal magnetic camera, comprising:
a channel comprising a proximal portion and a distal portion, wherein the distal portion is configured to retain an internal magnetic camera; and
a plunger arranged in the channel, wherein the plunger includes a receiver at a distal end configured to engage the internal magnetic camera, wherein the plunger is configured to slide relative to the channel, and wherein the plunger dislodges the retained internal magnetic camera from the channel when the plunger moves distally relative to the channel.

26. The introducer of claim 25, further comprising a longitudinal slot in the plunger extending from the distal end of the plunger.

27. The introducer of claim 26, further comprising a seal covering the longitudinal slot in the plunger.

28. The introducer of claim 27, further comprising a longitudinal slit in the seal.

29. The introducer of claim 27, further comprising at least one aperture in the seal.

30. The introducer of claim 25, wherein the distal portion of the channel comprises an arcuate cross-section.

31. The introducer of claim 1, further comprising a heating element that is engageable with the shaft, the heating element heating the shaft and the internal magnetic camera retained therein when the heating element is engaged with the shaft.
